(19) United States
(12) Patent Application Publication
(10) Pub. No.: US 2014/0186909 A1
(43) Pub. Date: Jul. 3, 2014

(54) Flexible Photobioreactors, Systems and Methods

(71) Applicants: Kevin J. Calzia, Maynard, MA (US); Max Brett Tuttman, Cambridge, MA (US); John E. Longan, Nashua, NH (US); Thomas A. Urbanik, Somerville, MA (US)

(72) Inventors: Kevin J. Calzia, Maynard, MA (US); Max Brett Tuttman, Cambridge, MA (US); John E. Longan, Nashua, NH (US); Thomas A. Urbanik, Somerville, MA (US)

(21) Appl. No.: 14/237,054

(22) PCT Filed: Aug. 1, 2012
(86) PCT No.: PCT/US2012/049150

§ 371 Date: Feb. 4, 2014

Related U.S. Application Data
(60) Provisional application No. 61/515,522, filed on Aug. 5, 2011.
(60) Provisional application No. 61/598,196, filed on Feb. 13, 2012.

(51) Int. Cl. C12M 1/00; C12N 1/12; C12P 1/00

(52) U.S. Cl. CPC C12M 21/02; C12P 1/00; C12N 1/12
USPC 435/134; 435/132; 435/160; 435/161; 435/166; 435/257.1; 435/292.1

(57) ABSTRACT

The present invention provides a photobioreactor for a phototrophic microorganism, and culture medium therefor, comprising a capsule, the capsule comprising an elongated body comprised of a first, flexible polymer film that is at least partially transparent to light of a wavelength that is photosynthetically active in a phototrophic microorganism and divided width wise into a plurality of adjacent channels, each channel having a major cross-sectional dimension that is not more than about one-third of the length of the elongated body, when the elongated body is inflated; and a fluid distribution structure coupled to the elongated body adapted for fluid communication with the plurality of adjacent channels that distributes a flow of culture medium amongst the plurality of channels, wherein the fluid distribution structure is comprised of a second, flexible polymer film.

### RELATED APPLICATIONS

This application claims the benefit of U.S. Provisional Application No. 61/515,552, filed Aug. 5, 2011 and U.S. Provisional Application No. 61/598,196, filed Feb. 13, 2012. The entire teachings of these applications are incorporated herein by reference.

### BACKGROUND OF THE INVENTION

One of the primary limitations of using photosynthetic microorganisms as a method of carbon dioxide sequestration or conversion to products has been the need for development of efficient and cost-effective growth systems. Open algal ponds up to 4 km² have been researched, which, while requiring low capital expenditures, ultimately have low productivity as these systems are also subject to a number of problems. Intrinsic to being an open system, the cultured organisms are exposed to a number of exogenous organisms that can be symbiotic, competitive, or pathogenic. Symbiotic organisms can change the culture organisms merely by exposing them to a different set of conditions. Opportunistic species can compete with the desired organism for space, nutrients, etc. Additionally, pathogenic invaders can feed on or kill the desired organism. In addition to these complicating factors, open systems are difficult to insulate from environmental changes including temperature, turbidity, pH, salinity, and exposure to the sun. Therefore, there is a need to develop a closed, controllable system for the growth of algae and similar organisms.

Other photobioreactor configurations aimed at improving the surface area to volume ratio to maximize conversion of sunlight and CO₂ to biomass or other products, such as algal oil, have been described in the literature (see, e.g., Pulz, O., “Photobioreactors: Production systems for phototrophic microorganisms,” Appl. Microbiol. Biotechnol (2001) 57:287-293). These reactors have distinct advantages compared to open raceway bioreactors with respect to controlling temperature, pH, and nutrients, and limiting contamination. A key limitation to their adoption has been the cost versus benefit as it relates to the product being produced. Whereas valuable products such as carotenoids have been produced in such photobioreactors, the production of biomass for fuels cannot be economically justified to date.

What is needed, therefore, is a photobioreactor capsule that is low cost, easily manufactured, and efficient for culturing light-capturing organisms.

### SUMMARY OF THE INVENTION

One embodiment of the present invention is a photobioreactor, which includes a capsule with a top wall and a bottom wall. One or both of the top wall and bottom wall are a flexible material (e.g., a thin polymer film) and the top wall is at least partially composed of a material that is at least partially transparent to light of a wavelength that is photosynthetically active in a phototrophic microorganism. A plurality of (e.g., two or more, three or more, five or more, eight or more) adjacent channels for enclosing microorganisms and culture medium are provided by sealing the top wall to the bottom wall to form seams. In this embodiment, each of the plurality of channels has a lay flat width between about 1 mm and about 2 m, more specifically, between about 15 mm and about 1 m or, yet more specifically, between about 10 mm and about 100 mm. In this embodiment, the capsule further includes a fluid distribution structure coupled to the plurality of adjacent channels adapted for fluid communication with the plurality of adjacent channels that distributes a flow of culture medium amongst the plurality of adjacent channels, wherein the fluid distribution structure is comprised of a flexible polymer film.

Other embodiments include one or more of the following variations. The seams can be formed by welding or heat sealing the top wall to the bottom wall. Each channel, when filled with microorganisms and culture medium, can have a substantially semi-circular, substantially circular, or substantially elliptical cross-section. The top wall and bottom wall can be made of a polymer or a polymer-composite film. The plurality of adjacent channels can have a length of about 100 meters and a width of about 1 meter. Alternatively, the plurality of adjacent channels of the photobioreactor can have a length of about 10 m to about 300 m. The capsule can have four or more channels for enclosing a phototrophic microorganism and culture medium therefor. A substantially even depth of microorganisms and culture medium can be provided across the channels and averages between about 5 mm and about 30 mm, between about 10 mm and about 30 mm, or between about 20 mm and about 30 mm. The bottom wall can be an abrasion resistant material. The plurality of channels can be of a thin-wall construction that couples to a circulation driver producing a flow of the microorganisms and culture medium through the channels.

Another embodiment of the invention is a photobioreactor for a phototrophic microorganism, and culture medium therefor, comprising a capsule comprising an elongated body having a length and a width and comprised of a flexible polymer film that is at least partially transparent to light of a wavelength that is photosynthetically active in a phototrophic microorganism, wherein the elongated body is divided widthwise into a plurality of adjacent channels, each having its major cross-sectional dimension that is not more than about one-third of the length of the elongated body, when the elongated body is inflated; and a fluid distribution structure coupled to the elongated body adapted for fluid communication with the plurality of adjacent channels that distributes a flow of culture medium amongst the plurality of channels.

Another embodiment of the invention is a photobioreactor for a phototrophic microorganism, and culture medium therefor, comprising a capsule comprising a plurality of adjacent channels, each of the plurality of adjacent channels having an inflow end and an outflow end, the plurality of channels being in a substantially planar arrangement and being formed by bonding two or more polymer films collinearly and lengthwise in a flow direction, the bonding providing bonded seams; a fluid distribution channel formed across the inflow end of the plurality of channels and substantially perpendicularly to the flow direction, the fluid distribution channel having a fluid inlet at an end of the fluid distribution channel, being in fluid communication with the plurality of channels, being adapted to distribute a flow of culture medium amongst the plurality of channels, and being formed by a polymer film; and a fluid collection channel formed across the outflow end of the plurality of channels and perpendicularly to the flow direction, the fluid collection channel having a fluid outlet at an end of the fluid collection channel, being in fluid communication with the plurality of channels, and being formed by a polymer film, wherein the two or more polymer films are at least partially transparent to light of a wavelength that is photosynthetically active in the phototrophic microorganism.

Another embodiment of the invention is a method of culturing a phototrophic microorganism using a photobioreactor or capsule of the invention.

Another embodiment of the invention is a method of producing a carbon-based product using a photobioreactor or capsule of the invention.

The photobioreactor capsules disclosed herein can maintain an environment that allows for microorganisms to operate at a high productivity by maximizing the surface area of the culture medium that the microorganisms are suspended in to increase photon absorption and maintaining a thin culture medium layer, which also increases photon absorption and provides for short light-dark cycle times and efficient heating ramp up of the culture medium. By breaking the width of the photobioreactor capsule up into several smaller channels or tubes that, when pressurized, inflate to smaller major cross-sectional dimensions or diameters, the depth of the culture medium can be decreased and the ratio of the major surface area to the culture depth can be increased, providing for increased photon absorption. In addition, the fluid distribution structures disclosed herein are adapted to reduce hoop stress or load on a capsule film or seams, thereby increasing the factor of safety of the capsules and/or enabling the capsules to be used under more challenging conditions, such as higher pressures and/or flow rates, with a decreased incidence of failure.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a perspective view of and illustrates a capsule constructed in accordance with an exemplary embodiment of the invention.

FIG. 2A is a top profile view diagram illustrating a capsule constructed in accordance with an exemplary embodiment of the invention.

FIG. 2B is a cross-sectional diagram along line A in FIG. 2A and illustrates a capsule constructed in accordance with an exemplary embodiment of the invention.

FIGS. 3A, 3B, and 3C are top profile view diagrams and illustrate exemplary fluid distribution structures coupled to elongated bodies (not shown in full) and constructed in accordance with exemplary embodiments of the invention.

FIGS. 4A, 4B, 4C, 4D, 4E, 4F, and 4G are top profile view diagrams and illustrate exemplary fluid distribution structures coupled to elongated bodies (not shown in full) and constructed in accordance with exemplary embodiments of the invention.

FIGS. 5A, 5B, and 5C are top profile view diagrams and illustrate exemplary fluid distribution structures coupled to elongated bodies (not shown in full) and constructed in accordance with exemplary embodiments of the invention.

FIG. 5D is a top profile diagram and illustrated an exemplary capsule constructed in accordance with exemplary embodiments of the invention.

FIG. 6 is a graph and shows the calculated hoop stresses associated with various channel diameters of an unrestrained 6 mil film capsule as a function of internal pressure.

FIGS. 7A and 7B are cross-sectional diagrams and illustrate a thin-film capsule inflated (FIG. 7A) and deflated (FIG. 7B).

FIG. 8 is a schematic diagram and shows a photobioreactor including an external pressure plate constraining a fluid distribution structure coupled to an elongated body (not shown in full).

FIG. 9 is a schematic diagram and illustrates a top plate and a bottom plate of a pressure plate from several perspectives.

FIG. 10 is a diagram and illustrates a 22-channel capsule having an integrated header.

FIG. 11 is a bar graph and shows the average calculated channel flow velocity in a capsule of FIG. 10 having a header diameter of 50 mm, 75 mm or 100 mm as a function of channel number (channels were numbered starting with the channel nearest the fluid inlet).

FIG. 12 is a schematic diagram and shows an exemplary C-tube assembly for use with a capsule, such as that depicted in FIG. 10, from several perspectives.

### DETAILED DESCRIPTION OF THE INVENTION

A description of preferred embodiments of the invention follows. The relevant teachings of all patents, published applications and references cited herein are incorporated by reference in their entirety. The following explanations of terms and methods are provided to better describe the present invention and to guide those of ordinary skill in the art in the practice of the present invention. As used herein, “comprising” means “including” and the singular forms “a” or “an” or “the” include plural references unless the context clearly dictates otherwise. For example, reference to “comprising a phototrophic microorganism” includes one or a plurality of such phototrophic microorganisms. The term “or” refers to a single element of stated alternative elements or a combination of two or more elements, unless the context clearly indicates otherwise.

Unless explained otherwise, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are illustrative only and not intended to be limiting. Other features of the invention are apparent from the following detailed description and the claims.

### Reactor Capsule

A first embodiment of the invention is a photobioreactor for a phototrophic microorganism, and culture medium therefor, comprising a capsule, the capsule comprising an elongated body having a length and a width and comprised of a first, flexible polymer film that is at least partially transparent to light of a wavelength that is photosynthetically active in a phototrophic microorganism, wherein the elongated body is divided widthwise into a plurality of adjacent channels, each having a major cross-sectional dimension that is not more than about one-third of the length of the elongated body, when the elongated body is inflated; and a fluid distribution structure coupled to the elongated body adapted for fluid communication with the plurality of adjacent channels that distributes a flow of culture medium amongst the plurality of channels, wherein the fluid distribution structure is comprised of a second, flexible polymer film. In a particular embodiment, each of the plurality of adjacent channels is substantially equivalent to every other channel. In another particular embodiment, the first flexible polymer film and the second flexible polymer film are the same.

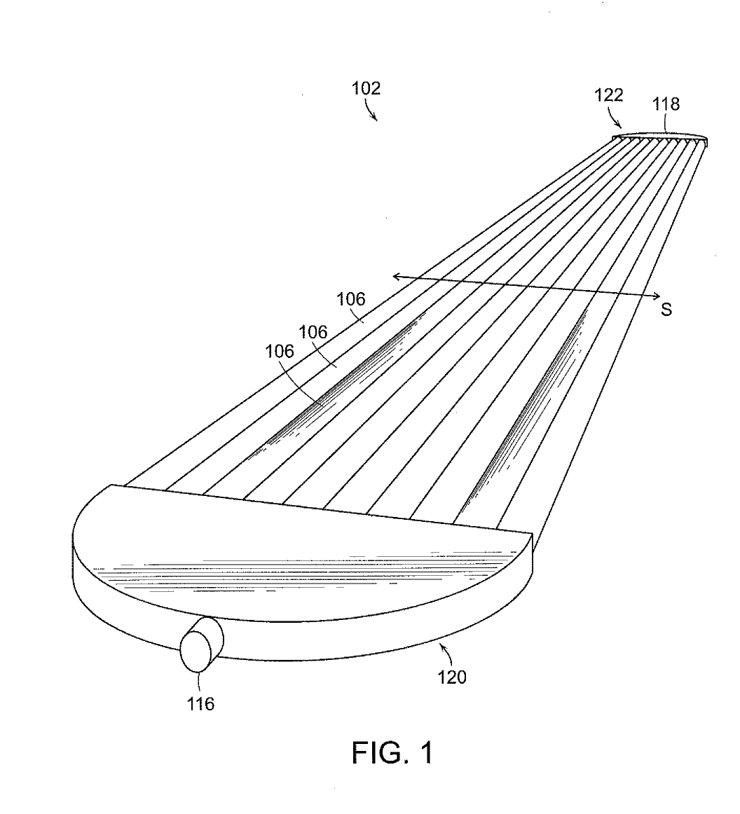

FIG. 1 is a perspective view of an exemplary capsule of the invention. Capsule 102 includes channels 106 of the elongated body coupled to inlet 116 via fluid distribution structure 120. Channels 106 are also coupled to outlet 118 via fluid distribution structure 122. In some embodiments, fluid distribution structures 120 and 122 are mirror images of one another (e.g., photobioreactor 102 is symmetrical around axis S). In other embodiments, fluid distribution structures 120 and 122 are not mirror images of one another (e.g., photobioreactor 102 is not symmetrical around axis S).

A second embodiment of the invention is a capsule comprising an elongated body comprising a first, flexible top wall composed at least partially of a material that is at least partially transparent to light of a wavelength that is photosynthetically active in a phototrophic microorganism, and a first, flexible bottom wall, wherein two or more channels for enclosing the phototrophic microorganism and culture medium therefor are provided by coupling the top wall to the bottom wall with a laying flat width of each two or more channels between about 15 mm and about 1 m; and a fluid distribution structure including a second, flexible top wall coupled to the first, flexible top wall and a second, flexible bottom wall coupled to the first, flexible bottom wall.

A third embodiment of the invention is a capsule, the capsule comprising an elongated body divided widthwise into a plurality of adjacent channels, the elongated body having a length of at least 50 m and a width of about 1 m to about 2 m, inclusive, and including a first top wall and a first bottom wall sealed lengthwise to form two or more seams that form the plurality of adjacent channels; and a fluid distribution structure including a second top wall coupled to the first top wall and a second bottom wall coupled to the first bottom wall, wherein the first and second top walls are formed from a first single piece of a flexible polymer composite film and the first and second bottom walls are formed from a second single piece of the flexible polymer composite film, the flexible polymer composite film is at least partially transparent to light of a wavelength that is photosynthetically active in a phototrophic microorganism, and each channel has a major cross-sectional dimension of between about 25 mm and about 50 mm, inclusive.

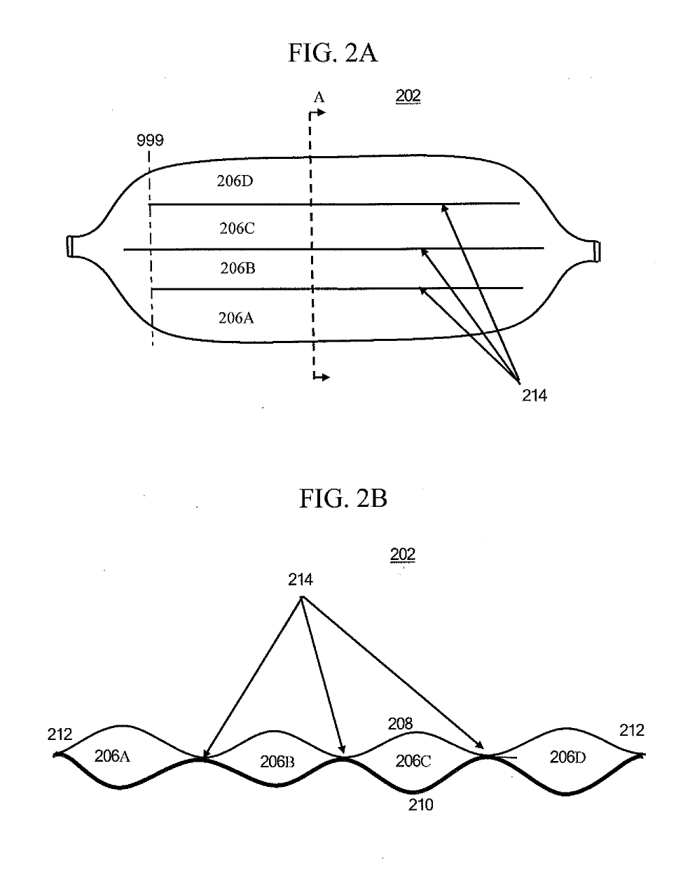

In one aspect of the above embodiments, the top and bottom walls of the elongated body are sealed lengthwise at even intervals to form two or more evenly spaced seams. In the capsule depicted in FIGS. 2A and 2B, channels 206A-D are formed by seams 214 welded lengthwise between top sheet 208 and bottom sheet 210. Seams can be produced using a variety of known welding techniques, for example, conductive, laser, microwave, ultrasonic weld, or chemical welding devices and methods. Polymer melts or reactive chemistry can also be used to form seals. Other materials can be used to seal the top and bottom sheets to form seams, for example, adhesives (e.g., extruded adhesives), laminates or fasteners (e.g., an external clamshell).

The phototrophic microorganism contained in the photobioreactor capsules of the invention require light for growth and/or the production of carbon-based products of interest. Therefore, the capsules and, in particular, channels 206A-D in FIGS. 2A and 2B are adapted to be at least partially composed of a material that is at least partially transparent to light of a wavelength that is photosynthetically active in the phototrophic microorganism.

Thus, another aspect of the above embodiments is an elongated body including a top wall composed of a flexible polymer film at least partially transparent to light of a wavelength that is photosynthetically active in a phototrophic microorganism and a bottom wall, wherein the top and bottom wall are sealed lengthwise to form one or more seams that form the plurality of channels in the elongated body. In a particular aspect of this aspect of the above embodiments, the top wall and the bottom wall of the elongated body are both composed of the same flexible polymer film.

FIG. 2A is a top profile diagram of an exemplary capsule of the invention. FIG. 2B is a cross-sectional diagram along line A of the capsule depicted in FIG. 2A. Capsule 202 is provided by a thin-film, flexible polymeric material. In the capsule depicted in FIGS. 2A and 2B, capsule 202 is formed by welding top sheet 208 and bottom sheet 201 lengthwise along both outside edges 212 to form seams. Capsule 202 includes a plurality of channels (206A, 206B, 206C, and 206D) and is adapted to allow cultivation in culture medium of a phototrophic microorganism.

Phototrophic microorganisms growing in photobioreactors can be suspended or immobilized. Typically, the average depth of the layer of culture medium is between about 5 mm and about 30 mm, between about 10 mm and about 15 mm or, preferably, between about 10 mm and 30 mm or between about 20 mm and about 30 mm. By inflating capsule 202 to predetermined amounts with a number of channels across the width of capsule 202, a desired depth of the layer of phototrophic microorganisms can be achieved. A substantially even layer can have various depths ranging from about 5 mm to about 30 mm for a substantial surface area portion of the layer, or from about 10 mm to about 15 mm.

In some embodiments, a capsule can include blown films that are manufactured as a collapsed tube. The blown films can have the shape of multiple channels or include individual channels of blown films sealed together. The sealing can be provided by using a variety of known welding techniques, for example, conductive, laser, microwave, ultrasonic weld, or chemical welding devices and methods.

Top sheet 208 need not include structural support elements. In one embodiment, the internal pressure within capsule 202 supports top sheet 208 above the culture medium and microorganisms. Top sheet 208 can be collapsed onto bottom sheet 210 when the culture medium, microorganisms and gases are removed from capsule 202.

Capsule 202 is not limited to four channels and can have as many or as few as desired according to the intended design constraints of capsule 202. These design constraints can include, for example, the major cross-sectional dimension of each channel, end region geometry, total width of the capsule, flow characteristics, mechanical stress on the capsule, and/or other characteristics of capsule 202. Channels 206A-D are also not limited to a straight path as shown in FIGS. 1, 2A and 2B. Embodiments can utilize curved seams 214 that produce winding paths or channels with varying widths. Channels 206A-D can also be a variety of different shapes and sizes. Each channel 206A-D of a photobioreactor can be of a different shape and dimension. Typically, however, in capsule 202 with a plurality of channels 206A-D, the channels are substantially equivalent (e.g., produce similar or identical amounts of the microorganism, produce similar or identical amounts of a carbon-based product, support similar or identical flow rates, support similar or identical culture depths, are of similar or identical shape and dimensions, or a combination thereof), for example, channels positioned in parallel with substantially longer channel length than major cross-sectional dimension (e.g., the major cross-sectional dimension is not more than one-third the length when the channels are inflated). Various capsule cross-sections are suitable, for example, substantially circular or half-elliptical. Preferably, the capsule cross-section is substantially half-elliptical or rectangular.

The capsule is not limited to both top and bottom sheets being of flexible material. In one embodiment, the bottom sheet can be a relatively rigid material sealed to a top sheet of a flexible material along the edges and lengthwise to form seams, as previously described, that form channels. Typically, however, the capsule can be enclosures (e.g., bags) welded from thin polymeric films.

Top sheet 208 can be transparent for light of a wavelength that is photosynthetically active in a phototrophic microorganism. This can be achieved by proper choice of the material, for example, thin-film material for top sheet 208 to allow light to enter the interior capsule. Bottom sheet 210 can be provided by a thin-film material enclosure, typically made from a polymeric material with abrasion resistance. In applications in which bottom sheet 210 rests directly on the ground, the bottom sheet can include a polymeric material of greater thickness, multiple layers, and/or additional features that can prevent abrasions or tears due to contact with coarser material, such as, gravel, or other objects on the ground. These features can include, but are not limited to, impregnated mesh and/or rubberized or other protective coatings. Bottom sheet 210 can also incorporate a reflective internal surface to allow light that has passed through capsule 202 to be reflected back through capsule 202 to allow the phototrophic microorganism to capture more light. Embodiments can include top sheet 208 and bottom sheet 210 being encapsulated with additional independent top and bottom enclosures that provide the abrasion resistance and/or other characteristics that protect capsule 202 from the external environment while allowing passage of a wavelength of light for photosynthetic activity.

In one aspect of the above embodiments of the invention, the top wall consists essentially of a first flexible polymer film and the first flexible polymer film has a thickness of less than 500 micrometers.

An inflating process to predetermined amounts can occur before, during, or after the introduction of the culture medium and/or microorganisms being introduced into capsule 202. The predetermined amount can be designated by a specific pressure (e.g., an inlet pressure of greater than 0.5 psi, greater than 2.0 psi, greater than 4 psi, greater than 6 psi, or greater than 10 psi), volume or other unit. The predetermined amount is not limited to a specific quantity but can include algorithms and/or increments of various amounts including, but not limited to, sequential increases and/or decreases.

The capsule described herein can be placed on the ground or floated on water such that top sheet 208 is directed upwards and bottom sheet 210 can be placed on the ground or water. Alternatively, the capsule(s) can also be placed above the ground and can utilize solid support structures made of, for example, wood, metal, mesh or fabric. Other support structures can include, for example, stakes, anchors or cables attached to flaps along the edges of the capsule. In one exemplary embodiment, straps or loops are provided along both edges of the capsule. The straps or loops can be used in conjunction with stakes or anchors to secure the capsule to the ground or other surface. The straps or loops can be of the same material as bottom sheet 210, or of another material. The straps or loops can be molded into or adhered to seam 214 or coupled to holes provided in the center of seam 214 such that a seal is provided around the hole and within the seam. The support structures can be used to prevent rotation or movement of the capsule. The support structure can be used to prevent movement due to gravity, unintended external forces and/or inclement weather.

The photobioreactors can include, in addition to a capsule, a number of devices that can support the operation of the photobioreactors. For example, devices for flowing gases (e.g., carbon dioxide, air, and/or other gases), measurement devices (e.g., optical density meters, thermometers), inlets and outlets, and other elements can be integrated or operationally coupled to the capsule, Capsule 202 can include further elements (not shown) such as inlets and outlets, for example, for growth media, carbon sources (e.g., CO₂), and probe devices such as optical density measurement devices, thermostats, and thermometers.

Further, capsule 202 can be adapted to allow gas flow through various channels 206A-D. Gas (e.g., CO₂) flow can be co- or counterdirectional to liquid flow through the reactor. For example, in certain embodiments, the capsule is adapted to allow codirectional gas flow in one part of the capsule and counterdirectional gas flow in another part of the capsule. In other embodiments, one or more capsules in an array of capsules are adapted to allow codirectional gas flow, and one or more other capsules of the photobioreactor in an array of capsules are adapted to allow counterdirectional gas flow.

In additional embodiments, support straps or a sheet can be used to further allow for a desired shape of channels 206A-D. The support straps can be strips of polymeric material or cords across the width of channels 206A-D. As channels 206A-D are inflated, the support straps can be brought into tension providing more of a rectangular shape with a desired major cross-sectional dimension to channels 206A-D. The support straps or sheet can also be a mesh sheet or other sheet of material with various openings. The support straps can be incorporated in the manufacturing of the capsule design by placing the straps or threads between top and bottom sheets 208, 210 of thin film of capsule 202. The straps or threads can be coupled to the outer edges during an edge sealing process. The sealing process can weld or adhere the straps or threads between top and bottom sheets 208, 210.

A circulation driver can be used to provide a flow of culture medium in the capsules described herein. Exemplary embodiments can utilize one or more circulation drivers and/or can comprise a single directional flow. Circulation drivers are not limited to utilizing an induced circulation system. For example, gravity driven, tidal driven, air driven, thermally driven, or circulation systems that do not involve active contact with the material being circulated can also be used to provide circulation. Exemplary circulating drivers can also utilize active circulation devices, such as pumps or augers, that actively apply a contact and apply a force to the circulating material.

Exemplary embodiments of capsules described herein can utilize a collapsible tube constructed of polymer film approximately 1 meter wide by 100 meters in length, when inflated. The culture medium, microorganisms and gases can be introduced into a capsule via a port fitting at one end of the capsule and exit via a port fitting at the other end. An internal pressure can be created by the culture medium, microorganisms and gases. If the pressure is sufficiently high and the flexible tubes of the capsule are left unrestrained, the pressure can cause the collapsible tubes of the capsule to inflate into a long tubular shape. Each collapsible tube has a length and a major horizontal cross-sectional dimension. When the collapsible tubes are inflated, a major surface area of each tube can be calculated by multiplying the length by the major horizontal cross-sectional dimension.

The term “horizontal,” as used herein, is defined with respect to the surface which supports the capsule (e.g., the ground, the water). Thus, “horizontal” is essentially parallel to the surface that supports the capsule. If a capsule is supported by an inclined structure, “horizontal” is defined with respect to the incline. As previously explained, it is typically desirable to maximize the surface area and minimize the depth of the culture. Typically, the cross-section of each channel, when inflated with air at atmospheric pressure, can be semi-circular, of a short and wide rectangle, or elliptical. Also typically, depending on the fill level with culture medium and gas, operating pressure, and support for the flexible elongated body of the photobioreactor, the cross-sectional shape varies.

As used herein, “elongated body” refers to a body having a length, a width, and a height, wherein the length is substantially greater than the width, and the width is substantially greater than the height when the body is inflated. In some embodiments, the length is at least 3, at least 10, at least 50, about 100, or at least 100 times greater than the width. In some embodiments, the width is about 100, at least 100, about 40, about 20, or about 10 times greater than the height. In some embodiments, the length is about 100 times greater than the width, and the width is about 20 times greater than the height. In other embodiments, the length is about 10 to about 300 m, about 100 to about 300 m, about 50 to about 100 m, or about 100 m. In some embodiments, the width is about 1 and about 2 m or about 1 and about 1.5 m. In some embodiments, the height is about 10 to about 100 mm, about 30 to about 55 mm, about 40 to about 55 mm, or about 45 mm. In some embodiments, the length is about 100 m, the width is about 1 m, and the height is about 45 mm. In a particular embodiment, the elongated body has a length of about 100 to about 300 m, a width of about 1 to about 2 m, and a height of about 40 to about 55 mm. In an alternative particular embodiment, the length of the elongated body is at least 50 m and the height of each channel is between about 10 mm and 100 mm. However, any combination of a length, a width, and a height recited above is possible.

In some embodiments, the elongated body is rectangular. In other embodiments, each of the plurality of adjacent channels of the elongated body are substantially equivalent or, more specifically, are of similar or identical shape and dimensions. In yet other embodiments, the elongated body is rectangular and each of the plurality of adjacent channels of the elongated body are substantially equivalent or, more specifically, are of similar or identical shape and dimensions.

The flexible polymer film that forms the capsule, elongated body, and/or fluid distribution structure can be a homopolymer, copolymer or even a multi-layered composite polymer based film. For example, a photobioreactor capsule can also be composed of a multi-layer film composed predominately of polyethylene and nylon. In one version the photobioreactor capsule can be fabricated from two, separate multi-layer films that are laminated together and can be represented as
80 laminated|80 co-extruded,
   where the numbers represent the layer thickness in micrometers. One of the starting films is made via lamination from cast film and the other starting film is made via a co-extrusion blown film process. The film structure showing the composition of the two starting films can be represented as: (60 PE|5 adhesive|15 PA)|(15 PA|4.8 tie|25 PE|4.8 tie|8 PA|4.8 tie|25 PE), where PE is polyethylene, PA is polyamide and tie refers to a thin (e.g., less than about 5 microns) layer of a polymeric material used to join dissimilar polymers (e.g, polyethylene and polyamide or nylon), Typically, a tie layer contains chemical functionality that will react with one of the dissimilar polymers to form chemical bonds and is miscible with the other dissimilar polymer so there is entanglement of the polymer chains. In a specific embodiment of the film structure described above, the tie layer is a co-polymer of ethylene and maleic anhydride.

Seals can be used to restrain the flexible walls from inflating into one large channel. For example, seals can be formed by welding or heat sealing a top wall to a bottom wall lengthwise along the chamber, providing seams that form multiple individual channels along the length of the chamber. Alternatively, seals can be formed by a pressure plate (e.g., an external clamshell) that exerts adequate force on the flexible walls of the chamber to create a fluid-tight seal. A pressure plate can also be used to reinforce, for example, a portion of a welded seal (e.g., an end of the welded seal).

Another embodiment of the invention is a capsule comprising a plurality of adjacent channels, each of the plurality of adjacent channels having an inflow end and an outflow end, the plurality of channels being in a substantially planar arrangement and provided by two or more polymer films bonded collinearly and lengthwise in a flow direction; a fluid distribution channel formed across the inflow end of the plurality of channels and substantially perpendicularly to an intended flow direction, the fluid distribution channel having a fluid inlet at an end of the fluid distribution channel, being coupled to the plurality of adjacent channels, being adapted for fluid communication with the plurality of adjacent channels to distribute a flow of culture medium amongst the plurality of adjacent channels, and being formed by a second polymer film; and a fluid collection channel formed across the outflow end of the plurality of adjacent channels and perpendicularly to the intended flow direction, the fluid collection channel having a fluid outlet at an end of the fluid collection channel, being coupled to the plurality of adjacent channels, being adapted for fluid communication with the plurality of adjacent channels, and being formed by a third polymer film, wherein the two or more polymer films are at least partially transparent to light of a wavelength that is photosynthetically active in the phototrophic microorganism.

In one aspect of this embodiment, the length of each channel is at least about 50 m, each channel has an inflated diameter between about 10 mm and about 100 mm, inclusive, and the fluid distribution channel has an inflated diameter of about 50 mm and about 250 mm, inclusive.

In another aspect of this embodiment, the length of each channel is at least about 50 m, each channel has an inflated diameter between about 25 mm and about 50 mm, inclusive, and the fluid distribution channel has an inflated diameter of about 50 mm and about 125 mm, inclusive.

The present invention also provides a method of fabricating a photobioreactor capsule, the method comprising extruding a sheet of flexible polymer film to produce a first sheet of flexible polymer film and a second sheet of flexible polymer film; and coupling the first sheet of flexible polymer film to the second sheet of flexible polymer film to form an elongated body comprising a plurality of adjacent channels and a fluid distribution structure.

In some embodiments of the method of fabrication, the first sheet of flexible polymer film and the second sheet of flexible polymer film are the same and are a composite polymer film.

In some embodiments, the method of fabrication further comprises forming a series of barriers within the fluid distribution structure by heat sealing the first sheet of flexible polymer film to the second sheet of flexible polymer film.

In some embodiments of the method of fabrication, the first sheet of flexible polymer film is coupled to the second sheet of flexible polymer film by heat-sealing the first sheet of flexible polymer film to the second sheet of flexible polymer film or by an extruded sealing process.

Another embodiment is a photobioreactor capsule of the invention, formed by extruding a sheet of flexible polymer film to produce a first sheet of flexible polymer film and a second sheet of flexible polymer film, and coupling the first sheet of flexible polymer film to the second sheet of flexible polymer film to form the elongated body comprising the plurality of adjacent channels and the fluid distribution structure.

### Fluid Distribution Structures

When culture medium is introduced into a channeled capsule without a fluid distribution structure, it is typical that the flow into the channels is not uniform. One method to address this potential problem is to provide a fluid distribution structure. In some embodiments, the fluid distribution structure is adapted to reduce hoop stress or the load on the capsule film or seams. A fluid distribution structure can be in the form of an asymmetrical or symmetrical fluid distribution structure, an integrated header, or an in-line distribution region.

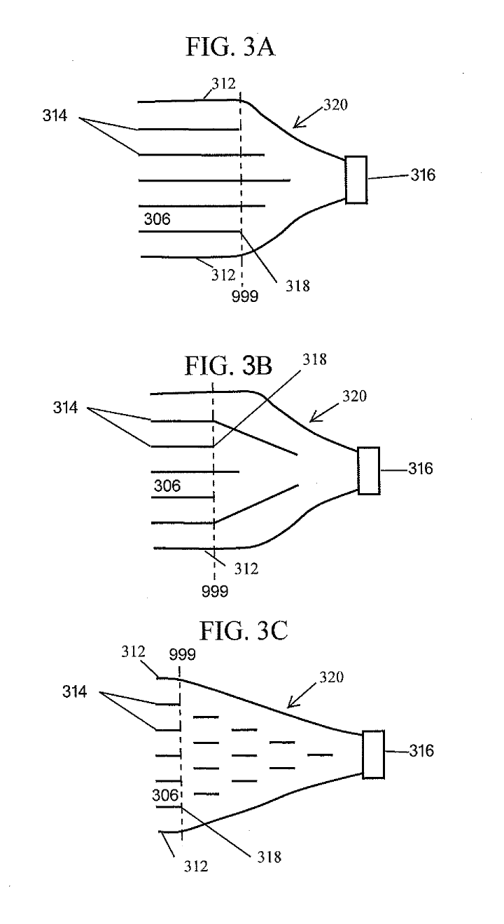

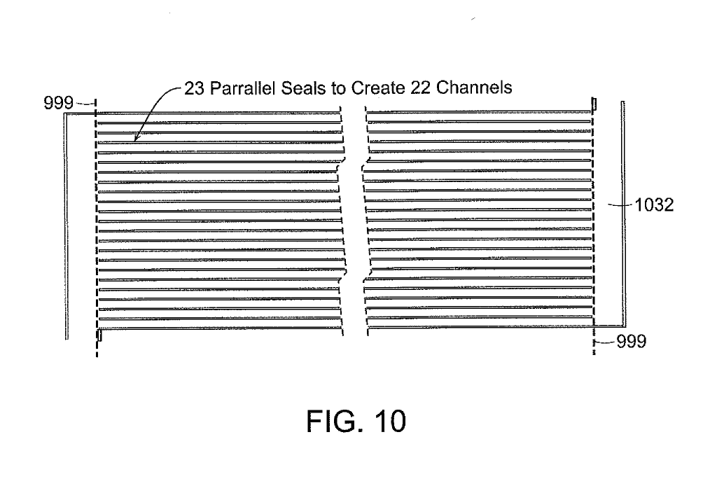

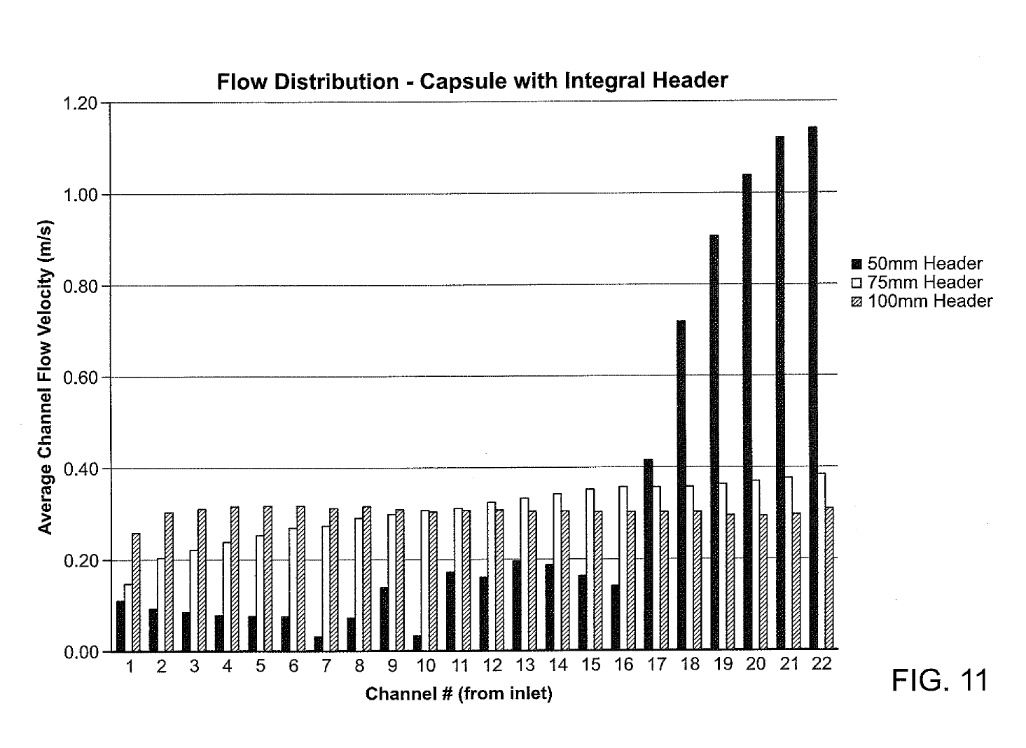

A fluid distribution structure can be designed to effect fluid distribution in several ways. For example, a fluid distribution structure can include one or more barriers to effect fluid distribution (as illustrated, for example, in FIGS. 3A-3C, 4A-4G, and 5A-5D). The shape, for example the tapered shape, of the fluid distribution structure can also effect fluid distribution, (as illustrated, for example, in FIGS. 1, 3A-3C, 4A-4G, and 5A-5D). A change in a dimension of a fluid distribution structure can also effect fluid distribution, as in the case of the integrated header depicted in FIG. 10, for example, in which the inflated diameter of the integrated header affects the distribution of the culture medium (see FIG. 11). Fluid distribution can also be effected by any combination of the above designs. For example, the fluid distribution structures illustrated in FIGS. 3A-3C, 4A-4G, and 5A-5D effect fluid distribution by including one or more barriers and by being tapered.

As used herein, “symmetrical fluid distribution structure” means a region of a photobioreactor capsule designed to support distribution (preferably, even distribution) of a flow of culture medium amongst a plurality of channels using a symmetrical arrangement of barriers.

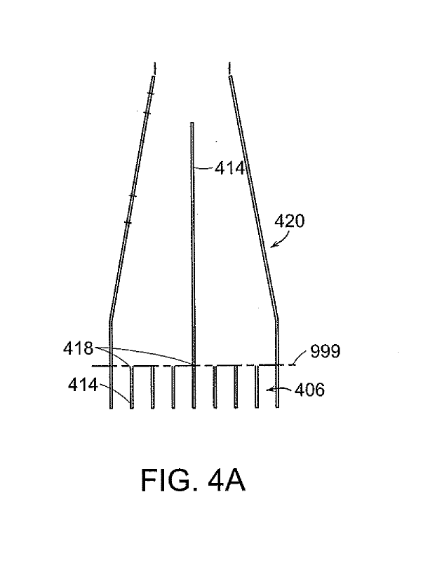

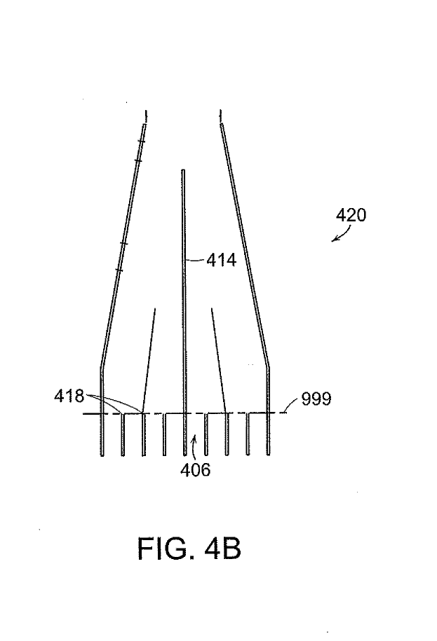

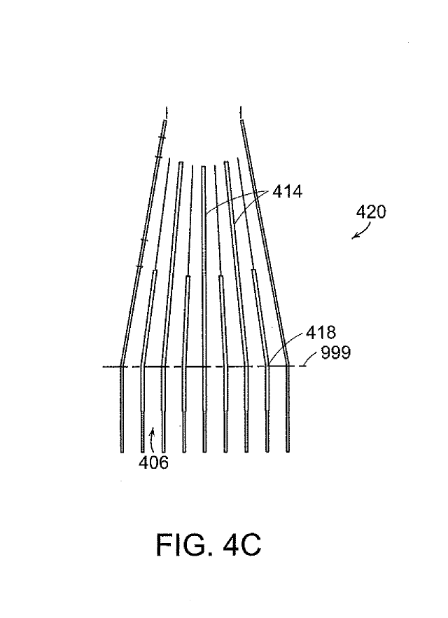

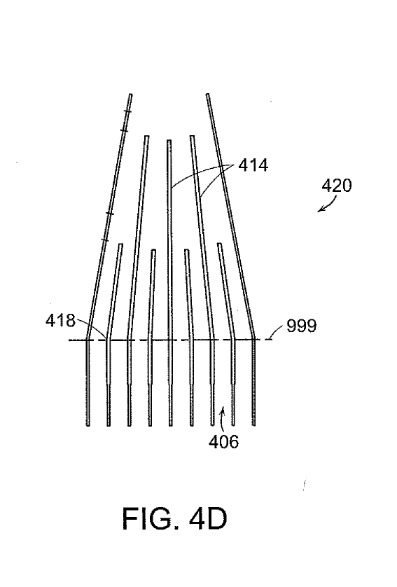

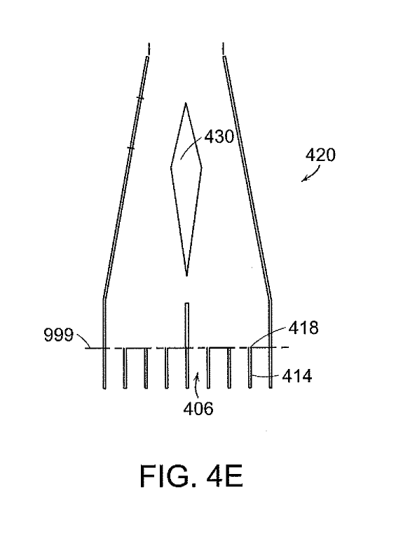

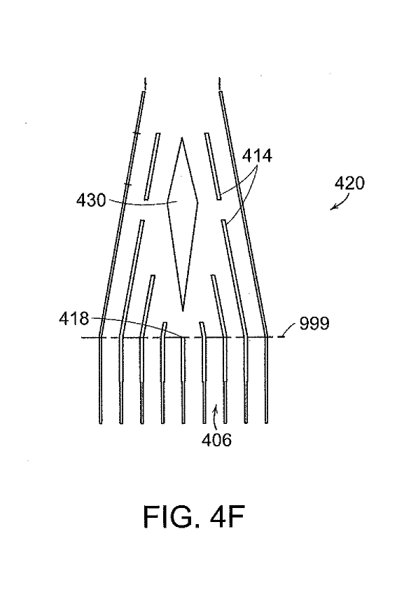

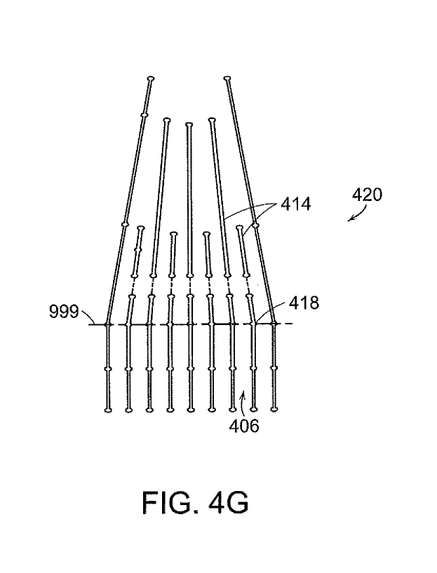

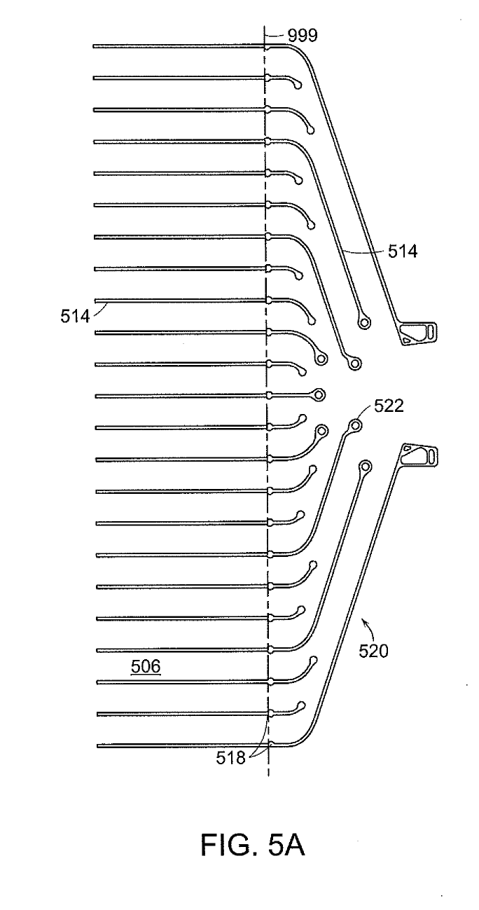

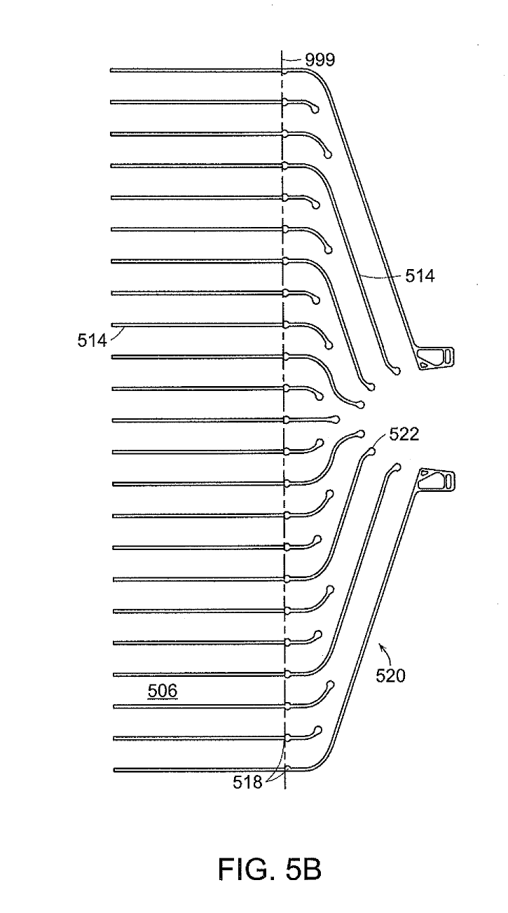

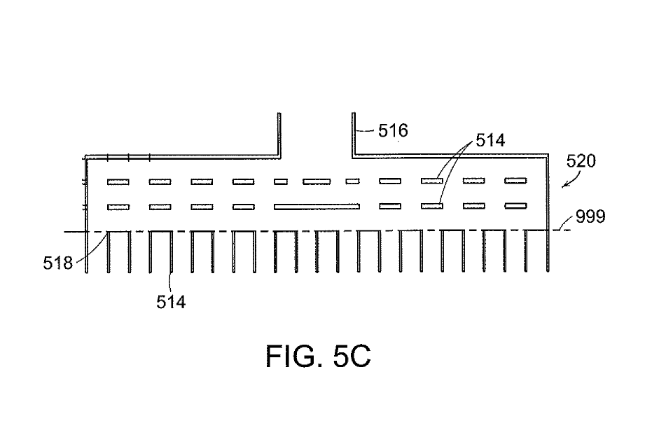

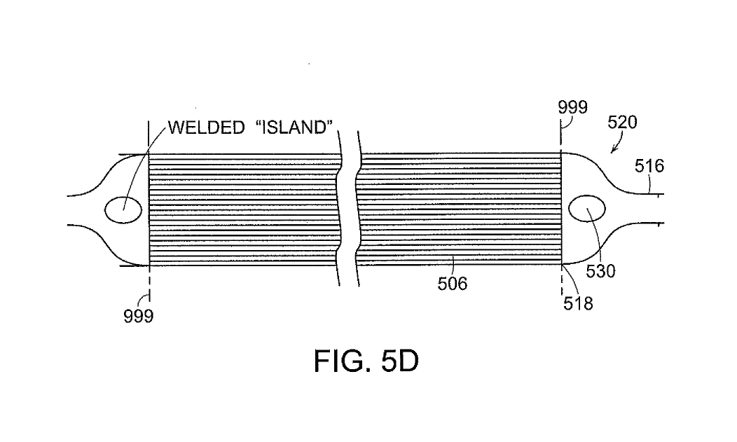

A barrier can be a seam formed in the interior of the fluid distribution structure of the capsule, such as the diamond depicted in FIG. 4E or the welded island depicted in FIG. 5D.

FIG. 10 illustrates an example of a 22-channel capsule of the invention with a fluid distribution structure in the form of an asymmetric integrated header 1032. In a capsule with an asymmetric integrated header, asymmetries in the header geometry may lead to unequal pressure drops along each flow path and thus uneven flow distribution. These pressure drops can be associated with the flow along the length of the header or with the transition from header to the channels. Flow traveling to channels furthest from the header inlet experience additional pressure drop along the length of the header, while flow traveling to the channels closest to the inlet undergo additional pressure drop associated with the sharp turn of high velocity flow. The undesirable effects of these asymmetric pressure drops can be mitigated if the symmetric pressure drops along each channel are significantly larger than the pressure drops along the length of the header or with the transition from header to channel, resulting in a similar total pressure drop for each flow path.

As used herein, “integrated header” means a region of a photobioreactor capsule designed to support distribution of a flow of culture medium amongst a plurality of channels by supporting a substantially equal pressure drop along each flow path. “Flow path” is any one of several paths liquid can take through a photobioreactor capsule of the invention. (e.g., from the inlet to the fluid distribution structure through a particular channel to the outlet of the photobioreactor capsule). An integrated header can be asymmetric (e.g., as shown in FIG. 10). An integrated header can also be tapered (e.g., the inflated diameter at one end of an asymmetric integrated header is 4 inches and is 2 inches at another end of the asymmetric integrated header). In some embodiments, an integrated header includes one or more barriers. Alternatively, in other embodiments, an integrated header is barrier-free (i.e., without barriers within the integrated header).

The photobioreactor capsules of the invention include a fluid distribution structure coupled to the plurality of adjacent channels or elongated body adapted for fluid communication with the plurality of adjacent channels that distributes a flow of culture medium amongst the plurality of adjacent channels. The flow of culture medium in each of the plurality of channels in the photobioreactors of the invention is typically co-directional. In some embodiments, the fluid distribution structure includes one or more barriers (e.g., at least 3, at least 5, at least 10, or at least as many barriers as there are seams separating channels of the elongated body (for example, barriers that are an extensions of the respective channel seams)) that support distribution of and, preferably, evenly distributes, the flow of culture medium amongst the plurality of channels. In some embodiments, the fluid distribution structure includes a top wall and a bottom wall, and the barriers are provided by sealing a portion of the top wall to a portion of the bottom wall. In some embodiments, the fluid distribution structure includes a top wall and a bottom wall that are sealed to one another to form the fluid distribution structure.

In a preferred embodiment of the invention, the fluid distribution structure includes one or more barriers that divide the flow of culture medium into two or more sub-flows, for example, a channel flow (e.g., the flow of culture medium in each of the plurality of adjacent channels in a capsule).

In some embodiments, the top wall of the elongated body and the top wall of the fluid distribution structure, together, are formed from a single piece or sheet of flexible polymer film, and the bottom wall of the elongated body and the bottom wall of the fluid distribution structure, together, are formed form a second, single piece or sheet of flexible polymer film. Alternatively, the top wall of the elongated body is connected to the top wall of the fluid distribution structure and the bottom wall of the elongated body is connected to the bottom wall of the fluid distribution structure. In a more specific embodiment, the top wall of the elongated body is sealed to the top wall of the fluid distribution structure and the bottom wall of the elongated body is sealed to the bottom wall of the fluid distribution structure.

Another method that can be used alone or in combination with the fluid distribution structure to create substantially uniform flow is to create breaks or stagger the channels along the length of the reactor channel. If the flow is not evenly distributed at the entrance to the channels, the breaks can be used to aid in re-distributing the flow further along the length of the capsule.

By creating channels along the length of the capsule, the width of the capsule decreases due to material being restrained in the channel seam. This can cause wrinkling and pinching of the film at the angled ends, possibly limiting flow of the culture or gases. To avoid this, the channel geometries at the entrance and exit region can be optimized. Several possible methods of doing this are shown, for example, in FIGS. 3-5. The channels can allow for substantial organism productivity, decreased stress in the film, continuous flow along the channels to supply gases and mixing, and/or allow for cleaning and sterilization of the capsule, among other advantages.

Distribution can be measured by comparing the volumetric flow of culture medium in a channel to the average flow of culture medium across all the channels. In some embodiments, the percent difference between a flow of culture medium in a channel and the average flow of culture medium across all the channels is less than or equal to 50%. Preferably, there is substantially even distribution, so that the percent difference between a flow of culture medium in a channel and the average flow of culture medium across all the channels is, for example, less than or equal to 20%, less than or equal to 10%, less than or equal to 5% or less than or equal to 1%.

FIGS. 3-5 are top-profile diagrams of various illustrative in-line fluid distribution structures coupled to elongated bodies (not shown in full) in accordance with the present invention. As used herein, “in-line fluid distribution structure” refers to a fluid distribution structure in which the direction of the fluid as it enters the fluid distribution structure is substantially the same as the direction of the fluid as it flows through the channels. The distribution can be designed to provide a substantially even distribution between the channels or can be designed to favor flow to certain channels. A substantially even distribution allows for substantially even amounts to be dispersed to each of the channels.

For illustrative purposes only, dashed line 999 in FIGS. 2A, 3A-3C, 4A-4G, 5A-5D and 10 indicates the boundary between the elongated body and the fluid distribution structure.

FIGS. 3A, 3B, and 3C show exemplary 6-channel fluid distribution structures coupled to elongated bodies (not shown in full). Other embodiments have analogous barrier arrangements with fewer or more than six channels. As shown in FIGS. 3A, 3B, and 3C, entrance port or inlet 316 feeds two or more channels formed by seams 314. As shown in FIG. 3A, seams 314 between opposing ends provide six channels for enclosing microorganisms and culture medium. Each of the five seams 314 is progressively shorter lengthwise from the center of the photobioreactor to each of the opposing edges 312. As shown in FIG. 3B, seams 314 assist to disperse the flow from the center of entrance port 316. The flow is initially dispersed into three sub-flows and subsequently the center sub-flow is further dispersed into two additional sub-flows, which are each subsequently dispersed into two additional channel flows in channels 306. As shown in FIG. 3C, seams 314 provide alternating sequences of separated flows and un-separated flows. At each point of un-separated flow, the flow can be distributed into two or more sub-flows. The liquid flow passes out of fluid transition region 320 and into channels 306 at point 318.

FIGS. 4A, 4B, 4C, 4D, 4E, 4F, and 4G show exemplary 8-channel distribution structures coupled to elongated bodies (not shown in full). In FIGS. 4A, 4B, 4C and 4D, seams 414 split the liquid flow and distribute it amongst channels 406. The liquid flow passes out of fluid transition region 420 and into channels 406 at point 418. FIG. 4E utilizes void space 430 in the shape of a diamond to distribute the liquid flow through fluid distribution structure 420, past point 418 and into channels 406. FIG. 4F uses a combination of void space 430 and seams 414 in fluid distribution structure 420 to distribute the liquid flow into channels 406. FIG. 4F also utilizes separated and un-separated flows to distribute the liquid flow into channels 406. FIG. 4G, like FIG. 4F, employs both seams 414, and separated and un-separated flows to distribute the liquid flow into channels 406. Void space 430 in FIGS. 4E and 4F is formed by a large welded space in the shape of a diamond. However, any shape may be used to form a void space. For example, a void space can be diamond-shaped, circular, oval-shaped or triangular. In addition, a void space may be formed by removing the excess material from the center of the welded area, as long as the integrity of the capsule is maintained.

FIGS. 5A, 5B, and 5C show exemplary 22-channel distribution structures coupled to elongated bodies (not shown in full). FIG. 5D shows an exemplary 22-channel capsule. FIGS. 5A and 5B resemble FIG. 3B, but illustrate fluid distribution structures having more channels. In addition, each seam 514 in FIGS. 5A and 5B ends in a circular sealed portion that distributes the hoop stress around the circumference of seam end 522. Bulges 518 in seams 514 in FIGS. 5A and 5B indicate the position at which the transition region is sealed to channels 506 of the photobioreactor. Fluid distribution structure 520 depicted in FIG. 5C utilizes seams 514 oriented perpendicularly to the fluid flow to distribute the liquid. FIG. 5D shows fluid distribution structure 520 having oval-shaped void space 530 to distribute liquid into channels 506.

Therefore, one aspect of the first to third embodiments of the invention is a fluid distribution structure coupled to the elongated body and adapted for fluid communication with the plurality of adjacent channels that evenly distributes a flow of culture medium amongst the plurality of channels, wherein the fluid distribution structure is comprised of a second, flexible polymer film, and includes a top wall and a bottom wall that are sealed to one another to form one or more barriers that support distribution of and, preferably, evenly distributes, the flow of culture medium amongst the plurality of channels.

In a specific aspect of this aspect of the first to third embodiments of the invention, the one or more barriers form a continuous extension of one or more of the seams into the fluid distribution structure. More specifically, the one or more barriers forming a continuous extension extend one or more of the seams of the elongated body linearly and lengthwise into the fluid distribution structure. Yet more specifically, there is a plurality of barriers and the plurality of barriers forming a continuous extension of the seams extends one or more of the seams linearly and lengthwise into the fluid distribution structure such that the channels become progressively shorter from a central channel(s) toward an outermost channel(s). Alternatively, or more specifically, the plurality of barriers forming a continuous extension of the seams extend one or more of the seams linearly and lengthwise into the fluid distribution structure and are substantially parallel to one another and substantially parallel to the seams. Alternatively, the plurality of barriers forming a continuous extension of the one or more seams extends one or more of the seams linearly and lengthwise into the fluid distribution structure and includes one or more barriers that are substantially parallel to the seams and one or more barriers that are angled toward one another. More specifically, the plurality of barriers include one or more central barriers that are substantially parallel to the seams and one or more flanking barriers that are angled towards the one or more central barriers.

In another specific aspect of this aspect of the first to third embodiments of the invention, the one or more barriers form a continuous and lengthwise extension of one or more of the seams into the fluid distribution structure. More specifically, the one or more barriers that form the continuous and lengthwise extension of one or more of the seams into the fluid distribution structure are curved, bent, or linear, or a combination thereof. Yet more specifically, a plurality of barriers form a continuous and lengthwise extension of one or more of the seams into the fluid distribution structure and the plurality of barriers includes one or more central barriers parallel to the seams and one or more flanking barriers curved, bent, or angled towards the one or more central barriers.

In another specific aspect of this aspect of the first to third embodiments of the invention, the one or more barriers form a lengthwise extension of one or more of the seams into the fluid distribution structure. More specifically, the one or more barriers that form the lengthwise extension of one or more of the seams into the fluid distribution structure are curved, bent, or linear, or a combination thereof. Yet more specifically, a plurality of barriers form a lengthwise extension of one or more of the seams into the fluid distribution structure and the plurality of barriers includes one or more central barriers parallel to the seams and one or more flanking barriers curved, bent, or angled towards the one or more central barriers.

In another specific aspect of this aspect of the first to third embodiments of the invention, the one or more barriers form a discontinuous and lengthwise extension of one or more of the seams into the fluid distribution structure. More specifically, a plurality of barriers form a discontinuous and lengthwise extension of one or more of the seams into the fluid distribution structure and the plurality of barriers includes one or more central barriers parallel to the seams and one or more flanking barriers curved, bent, or angled towards the one or more central barriers.

In yet another specific aspect of this aspect of the first to third embodiments of the invention, the one or more barriers include a void space. Specifically, the void space can have a variety of shapes, such as a diamond, circle, oval or triangle. Alternatively, the one or more barriers include a void space in combination with the one or more barriers described in any of the above aspects of this embodiment of the invention.

In another specific aspect of this aspect of the first to third embodiments of the invention, the one or more barriers are arranged in a staggered pattern. Specifically, the one or more barriers are arranged to provide alternating sequences of separated channel flows and un-separated channel flows in the fluid distribution structure, whereby the one or more barriers distribute each un-separated channel flow into two or more channel flows.

Embodiments of the fluid distribution structure are not limited to the displayed patterns and can utilize various combinations and patterns not shown.

Hoop Stress

The internal pressure created by the culture medium, microorganisms and gases create a hoop stress in the capsule. The hoop stress is calculated by the following equation:

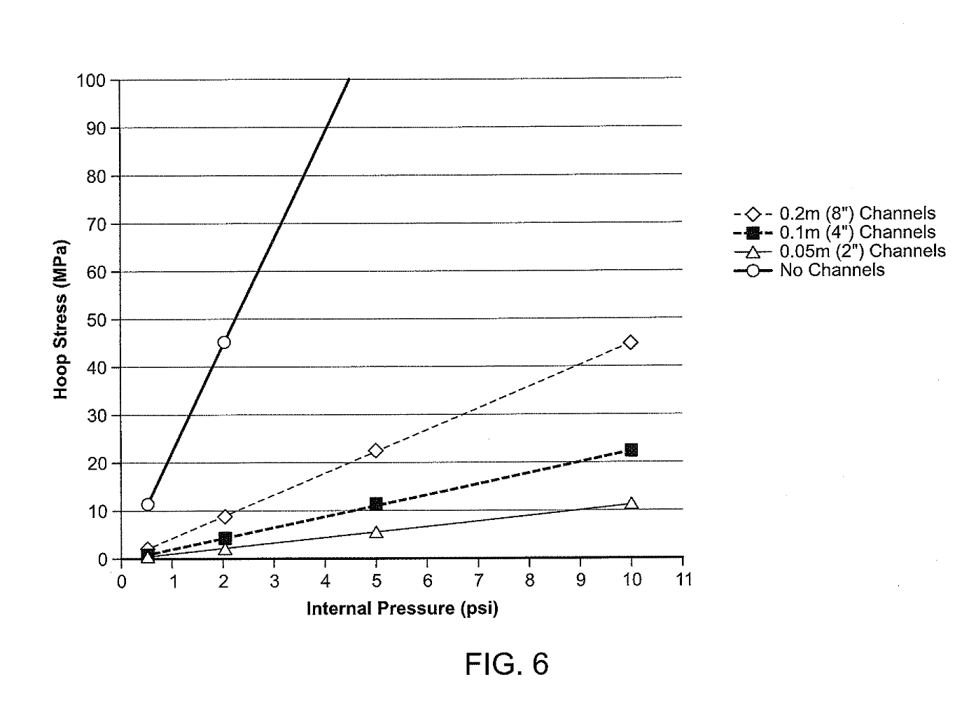

σₕ=Pd/2t,

where σₕ is the hoop stress, P is the pressure, d is the diameter, and t is the film thickness. By creating channels in the capsule, the diameter of the hoop is effectively decreased, thereby decreasing the hoop stress as shown in FIG. 6. This can allow for the use of lower strength, lower cost films or reducing the thickness of the film; higher operating pressures during operation at higher flow rates; aid in mixing, filling, and/or cleaning/sterilizing; and/or reducing the cost. Hoop stress can be converted to load in the film per linear inch of film using the following equation: load=Pd/2 or σₕt.

Table 1 shows the load in the film per linear inch of film (lb/in) at three different operating pressures assuming a 46 mm lay flat width (LFW) channel (29 mm or 1.14 in inflated diameter) or a 160 mm LFW header (102 mm or 4.02 in inflated diameter). The values are calculated following a hoop stress analysis. No factor of safety is applied to the allowable load in the film or seam. These values can be compared to the measured mechanical properties of the film or seam to verify that the film or seam is capable of handling the operating conditions.

As used herein, “factor of safety” means that the maximum load a film capsule can bear with less than 20% strain at a particular operating pressure is a factor greater than the maximum calculated load on the film capsule at the same operating pressure, as determined by a hoop stress analysis and conversion to load. In some embodiments, the factor is 3 times, 2 times, or 1.5 times. In other embodiments, the factor is at least 3 times, at least 2 times or at least 1.5 times. In some embodiments, the operating pressure is at least 2 psi, at least 4 psi, at least 6 psi or at least 10 psi. In some embodiments, the factor of safety is at least 3 at an operating pressure of 2 psi. In other embodiments, the factor of safety is at least 3 at an operating pressure of 4 psi. The maximum load a capsule can bear with less than 20% strain can be measured using a creep test.

In some embodiments, the one or more seams of the elongated body has a maximum load in a T-peel test that is greater than a maximum load of the flexible polymer film at less than 20% strain in a tensile test. In other embodiments, the one or more seams of the fluid distribution structure has a maximum load in a T-peel test that is greater than a maximum load of the flexible polymer film at less than 20% strain in a tensile test.

“Operating pressure,” as used herein, refers to the pressure as measured at the entrance of the capsule. In some embodiments of the invention, the one or more seams of the fluid distribution structure and/or the elongated body and the polymer film forming the fluid distribution structure and/or the elongated body are designed for operation at a pressure of at least 2 psi with a factor of safety of at least three. In a more particular embodiment, the one or more seams of the fluid distribution structure and/or the elongated body and the polymer film forming the fluid distribution structure and/or the elongated body are designed for operation at a pressure of at least 4 psi with a factor of safety of at least three.

TABLE 1
Calculated load on film or seam at various operating
pressures and inflated capsule diameters

| Operating | Inflated | Load on Film or Seam |
|---|---|---|
| Pressure | Diameter | (kg/15 mm) |
| (psi) | (mm) | *No factor of safety applied* |
| 2 | 29 | 0.31 |
| 4 | 29 | 0.62 |
| 6 | 29 | 0.93 |
| 2 | 102 | 1.07 |
| 4 | 102 | 2.15 |
| 6 | 102 | 3.22 |

One embodiment of the invention is a photobioreactor including a capsule and an external pressure plate (e.g., an external clamshell, a C-tube) designed to reduce the load on the capsule film or seam. A pressure plate can also be used to improve or manage fluid flow within the photobioreactor. Typically, a pressure plate is made of a material of greater stiffness than the capsule film (e.g., aluminum, rigid polyvinyl chloride, acrylic, fiber-reinforced composites).

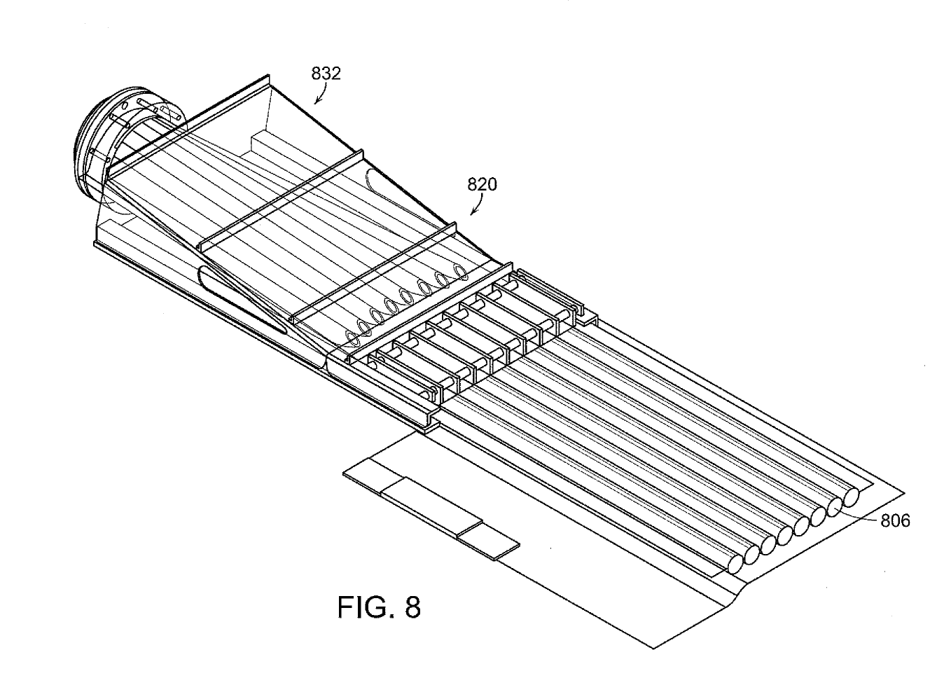

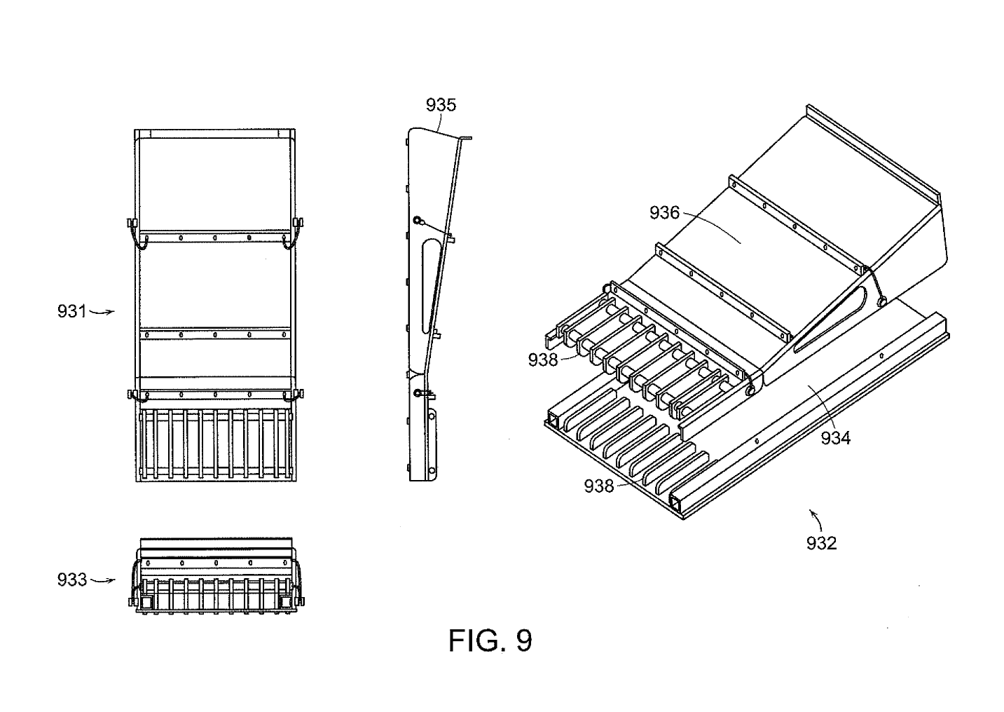

FIG. 8 is a schematic diagram and shows a photobioreactor capsule having channels 806 and fluid distribution structure 820, and external pressure plate 832. FIG. 9 is a schematic diagram and illustrates top plate 936 of pressure plate 932 from several perspectives: plan view 931, front view 933 and side view 935. Pressure plate 932 includes base plate 934, top plate 936, fins 938, and means for applying a substantially even amount of pressure through top and base plates 936 and 934 to fins 938. Base plate 934, top plate 936, and the means for applying pressure cooperate to reduce the load on the capsule film. In addition, when pressure plate 932 is assembled with the photobioreactor, fins 938 located on top plate 936 and bottom plate 934 align with one another and with the channel seal ends. Fins 938 apply pressure to the channel seal ends, thereby reinforcing the channel seams. Base and top plates 934 and 936 can be reinforced with additional beams to support the internal pressure of the photobioreactor without excessive deflection of the plates.

If the fluid transition region of the photobioreactor consists of relatively large areas without barriers or seals, then a pressure plate can allow for higher operating pressures by reducing hoop stress in the capsule film and isolating the ends of the channel seals from stress in those areas. In use, a fluid distribution structure is sandwiched between the top and base plates such that the fins on the top plate and the fins on the bottom plate align with the seal ends that form the boundaries of the channels. The plates are held closed with clamps or bolts, which apply pressure through the fins and squeeze the channel ends located between the base and top fins.

As pressure is increased within the photobioreactor, the plates limit the amount of deformation of the bioreactor. That is, the bioreactor will expand only to the extent allowed by the plates, thus limiting the unsupported periphery of the bioreactor to the space between the plates. This significantly reduces the hoop stress in the capsule film. By pinching the ends of the channel seals between the fins of the pressure plate, separation, tearing or peeling of the top and bottom sheets of the photobioreactor is prevented. An additional benefit of a pressure plate is that the fins help to shape the ends of the channels, thereby preventing wrinkles in the capsule film that could reduce culture flow into individual channels or lead to maldistribution of the culture medium.

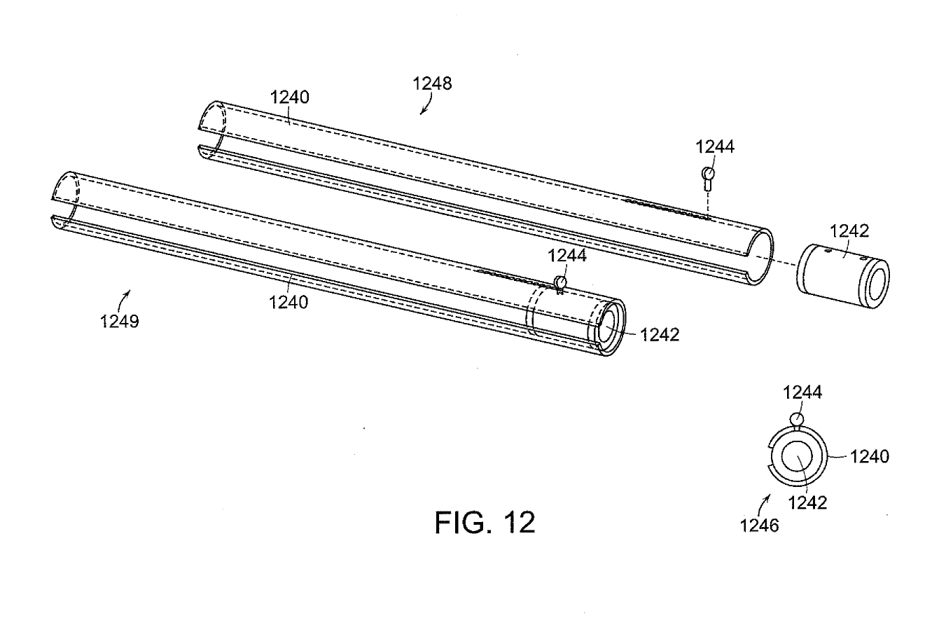

In some embodiments of the invention, a photobioreactor includes a capsule and a pressure plate, such as a C-tube. FIG. 12 is a schematic diagram and shows an exemplary C-tube assembly of the invention. Disassembled C-tube assembly 1248 includes C-tube 1240, pin 1244 and adjustable plug 1242. Pin 1244 and adjustable plug 1242 are designed to manage pressure and length of a fluid distribution structure in a capsule with a fluid distribution structure in the form of an integrated header. The depiction of assembled C-tube assembly 1249 shows adjustable plug 1242 inserted into C-tube 1240 and held at a desired location by pin 1244. Head-on view 1246 of an exemplary C-tube assembly of the invention also shows adjustable plug 1242 inserted into C-tube 1240 and held at a desired location by pin 1244.

### Channel Size

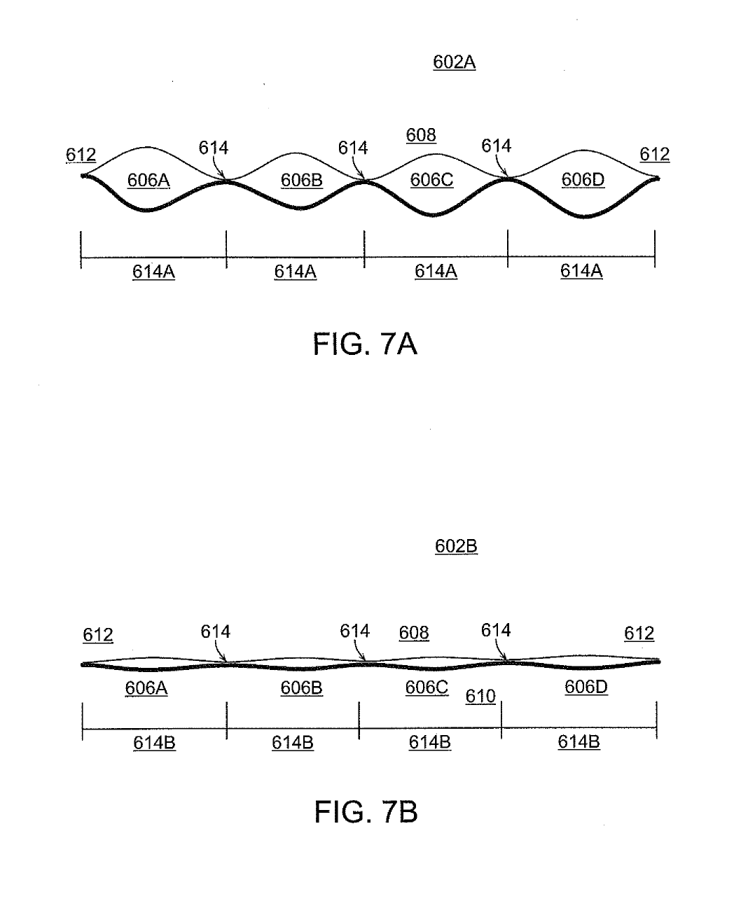

As shown in FIGS. 7A and 7B, a cross-sectional diagram of a thin-film capsule inflated 602A and deflated 602B is shown in accordance with the present invention, according to certain embodiments, Thin-film capsules 602A and 602B include one or more channels (606A, 606B, 606C, and 606D). As previously described in prior embodiments, top sheet 608 and bottom sheet 610 are sealed lengthwise along both outside edges 612 and seams 614.

One or more channels (606A, 606B, 606C, and 606D) are inflated and filled with the culture medium and a phototrophic microorganism, as shown in FIG. 7A. The culture medium of the phototrophic microorganism can be provided in a layer of liquid or semi-liquid that flows through each channel (606A, 606B, 606C, and 606D). As previously described, this layer may typically be between about 5 mm and about 30 mm deep, between about 10 mm and about 15 mm deep, between about 10 mm and 30 mm deep or between about 20 mm and 30 mm deep. Above this layer, a mixture of gases can be provided. This mixture of gases can be used to feed the microorganisms and/or be exhausted by the phototrophic microorganisms. A combination of gas pressure; thickness or volume of the layer of culture medium of the phototrophic microorganisms; and strength of top sheet 608, bottom sheet 610, and outside edges 612 and seams 614 can be used to determine inflated width 614A of channels 606A-D.

In some applications, it can be desirable to maximize the width of the channels (606A, 606B, 606C, and 606D) given the thickness or volume of the layer of culture medium of the phototrophic microorganisms and strength of the top sheet 608, the bottom sheet 610, and outside edges 612 and seams 614. As previously described, the maximum hoop stress σₕ can be determined for a given top sheet 608 and bottom sheet 610 strength. From the maximum hoop stress, a major cross-sectional dimension 614A can be determined. As used herein, “major cross-sectional dimension” or “major dimension” refers to the greatest cross-sectional dimension a channel assumes when it is inflated.

“Lay flat width,” as used herein, refers to the length of material between two seams of an uninflated and unfilled, or flat, channel formed from a top and a bottom sheet. Generally, the width of the top sheet and bottom sheet can be different, in which case the bottom perimeter between two seams can be different from the top perimeter between two seams. When the bottom perimeter between two seams differs in length from the top perimeter between two seams, the smaller perimeter corresponds to the lay flat width. FIG. 7B shows an embodiment in which channels 606A-D are formed by seams 614 located at 180 degrees from one another. Lay flat width 614B, as shown in FIG. 7B, may typically be between about 1 mm and about 2 m, between about 15 mm and about 1 m, between about 15 mm and about 300 mm, or between about 10 mm and 100 mm, Once the channels are filled with culture medium of the phototrophic microorganisms and gases during operation, lay flat width 614E shrinks to inflated diameter 614A of channels 606A-D. Table 2 shows exemplary lay flat widths and the corresponding inflated diameters, assuming 180-degree seams. “Diameter” is the major cross-sectional dimension of a channel whose cross-sectional shape approaches that of a circle.

TABLE 2
Channel Lay Flat Widths and Inflated Diameters

| Lay Flat Width (m) | Inflated Diameter (m) |
|---|---|
| 3.142 | 2.000 |
| 1.571 | 1.000 |
| 0.079 | 0.050 |
| 0.024 | 0.015 |
| 0.008 | 0.005 |
| 0.002 | 0.001 |

### Photobioreactor Biomass Productivity

The photobioreactor also provides methods to achieve organism productivity as measured by production of desired products, which includes cells themselves.

The desired level of products produced from the phototrophs in the photobioreactor can be of commercial utility. For example, the phototrophs in the photobioreactor use light, water and carbon dioxide to produce carbon-based products of interest (e.g., fuels, biofuels, biomass or chemicals) at about 5 to about 10 g/m²/day, in certain embodiments about 15 to about 42 g/m²/day and in more preferred embodiments, about 30 to 45 g/m²/day or greater.

The photobioreactor affords high areal productivities that offset associated capital cost. Superior areal productivities are achieved by: optimizing cell culture density through control of growth environment, optimizing CO₂ infusion rate and mass transfer, optimizing mixing to achieve highest photosynthetic efficiency/organisms, achieving maximum extinction of insulating light via organism absorption, and achieving maximum extinction of CO₂ and initial product separation.

In particular, the southwestern U.S. has sufficient solar insolation to drive maximum areal productivities to achieve about >25,000 gal/acre/year ethanol or about >15,000 gal/acre/year diesel, although a majority of the U.S. has insolation rates amenable to cost effective production of commodity fuels or high value chemicals.

Furthermore, CO₂ is also readily available in the southwestern U.S. region, which is calculated to support large scale commercial deployment of the invention to produce 25-70 g/m²/day ethanol, or 70 Bn gal/year diesel.

A fourth embodiment of the invention is a method of producing a carbon-based product in a photobioreactor or capsule of the invention, comprising culturing a phototrophic microorganism that produces the carbon-based product in a photobioreactor or photobioreactor capsule, the culturing comprising flowing culture medium containing the phototrophic microorganism into the fluid distribution structure at an operating pressure of greater than about 2 psi, thereby distributing culture medium to a substantially even depth across the plurality of adjacent channels; flowing the distributed culture medium through the plurality of adjacent channels; and providing carbon dioxide to the culture medium, whereby the phototrophic microorganism receives light of a wavelength that is photosynthetically active in the phototrophic microorganism.

A fifth embodiment of the invention is a method of producing a carbon-based product, comprising culturing a phototrophic microorganism that photosynthetically produces the carbon-based product in a photobioreactor, the photobioreactor comprising a capsule comprising an elongated body divided widthwise into a plurality of adjacent channels, the elongated body having a length of at least 50 m and a width of about 1 m to about 2 m, inclusive, and including a first top wall and a first bottom wall sealed lengthwise to form two or more seams that form the plurality of adjacent channels; and a fluid distribution structure including a second top wall coupled to the first top wall and a second bottom wall coupled to the first bottom wall, wherein the first and second top walls are formed from a first single piece of a flexible polymer composite film and the first and second bottom walls are formed from a second single piece of the flexible polymer composite film, the flexible polymer composite film is at least partially transparent to light of a wavelength that is photosynthetically active in a phototrophic microorganism, and each channel has a major cross-sectional dimension of between about 25 mm and about 50 mm, the method comprising flowing culture medium containing the phototrophic microorganism into and through the fluid distribution structure at an operating pressure of greater than about 2 psi, thereby distributing culture medium to the plurality of adjacent channels; flowing the distributed culture medium through the plurality of adjacent channels; providing carbon dioxide to the culture medium; providing light of a wavelength that is photosynthetically active in the phototrophic microorganism to the phototrophic microorganism as it flows through the plurality of adjacent channels, whereby the phototrophic microorganism produces the carbon-based product; and collecting the carbon-based product.

As used herein with respect to the methods of the invention, “providing light” includes locating a photobioreactor capsule in an area that receives light (e.g., sunlight) and positioning a light source such that at least a portion of the photobioreactor capsule receives light.

A sixth embodiment of the invention is a method of producing a carbon-based product, comprising culturing a phototrophic microorganism that photosynthetically produces the carbon-based product in the photobioreactor of any one of Claims 1-33, the method comprising flowing culture medium containing the phototrophic microorganism into and through the fluid distribution structure at an operating pressure of greater than about 2 psi, thereby distributing culture medium to the plurality of adjacent channels; flowing the distributed culture medium through the plurality of adjacent channels; providing carbon dioxide to the culture medium; providing light of a wavelength that is photosynthetically active in the phototrophic microorganism to the phototrophic microorganism as it flows through the plurality of adjacent channels, whereby the phototrophic microorganism produces the carbon-based product; and collecting the carbon-based product.

In one aspect of the fourth, fifth or sixth embodiment, the phototrophic microorganism is a genetically engineered phototrophic microorganism. In another aspect of the fourth, fifth or sixth embodiment, the operating pressure is greater than about 4 psi. In yet another aspect of the fourth, fifth or sixth embodiment, the culture medium is distributed to a substantially even depth across the plurality of adjacent channels. In yet another aspect of the fourth, fifth or sixth embodiment, the substantially even depth across the plurality of adjacent channels is about 5 to about 30 mm. In yet a more specific aspect of the fourth, fifth or sixth embodiment, the substantially even depth across the plurality of adjacent channels is about 20 to about 30 mm.

In another aspect of the fourth, fifth or sixth embodiment of the invention, the method comprises continuously flowing the culture medium containing the phototrophic microorganism into and through the fluid distribution structure.

As used herein, “continuously flowing” means flowing culture medium into and through a photobioreactor capsule for long periods of time (e.g., days, weeks, or months) at an operating pressure (e.g., at least about 2 psi, at least about 3 psi, or at least about 4 psi). “Continuously flowing” into the photobioreactor capsule (and, particularly, into the fluid distribution structure of a photobioreactor capsule) can be at a substantially constant flow rate or at a flow rate variable with time, and does not exclude stoppages or pauses in flow that may be necessary, for example, to perform maintenance or sampling on a photobioreactor. Typically, the photobioreactors and the photobioreactor capsules of the invention can be operated under continuous flow conditions without significant structural problems, for example, failure of the capsule film or seams.

Another embodiment of the invention is a method of culturing a phototrophic microorganism in the photobioreactor of any one of Claims 1-33, the method comprising flowing culture medium containing the phototrophic microorganism into and through the fluid distribution structure at an operating pressure of greater than about 2 psi, thereby distributing culture medium across the plurality of adjacent channels; flowing the distributed culture medium through the plurality of adjacent channels; providing carbon dioxide to the culture medium; and providing light of a wavelength that is photosynthetically active in the phototrophic microorganism to the phototrophic microorganism as it flows through the plurality of adjacent channels, thereby culturing the phototrophic microorganism.

In a specific aspect of this embodiment, the phototrophic microorganisms and culture medium are distributed to a substantially even depth across the plurality of adjacent channels by seams in the fluid distribution structure positioned to provide alternating sequences of separated channel flows and un-separated channel flows. Yet more specifically, each un-separated channel flow is re-distributed into two or more channel flows by additional seams in the fluid distribution structure.

In another specific aspect of this embodiment, the substantially even depth across the plurality of adjacent channels averages between about 5 mm to about 30 mm. In a more specific aspect of this embodiment, the substantially even depth across the plurality of adjacent channels averages between about 20 mm to about 30 mm.

In yet another specific aspect of this embodiment, the method comprises continuously flowing the culture medium containing the phototrophic microorganism into and through the fluid distribution structure.

### DEFINITIONS

Suitable phototrophic microorganisms can produce a carbon-based product and/or the phototrophic microorganism itself can be processed as feed stock for the production of a carbon-based product. Particularly suitable phototrophic microorganisms can be genetically engineered to produce a desired carbon-based product. Exemplary suitable phototrophic microorganisms are described in U.S. Pat. No. 7,919,303, U.S. Pat. No. 7,794,969, U.S. patent application Ser. No. 12/833,821, U.S. patent application Ser. No. 13/054,470, U.S. patent application Ser. No. 12/867,732, WO/2009/111513, WO/2009/036095, WO/2011/005548, WO/2011/006137 and WO/2011/011464.

“Carbon-based products of interest” include alcohols such as ethanol, propanol, isopropanol, butanol, fatty alcohols, fatty acid esters, ethyl esters, wax esters; hydrocarbons and alkanes such as pentadecane, heptadecane, propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals. More typical carbon-based products of interest are fuels (e.g., alcohols or alkanes). Even more typically, carbon-based products of interest are ethanol, propanol, isopropanol, butanol, terpenes, alkanes such as pentadecane, heptadecane, octane, propane, fatty acids, fatty esters, fatty alcohols, olefins or diesel.

As used herein, “light of a wavelength that is photosynthetically active in the phototrophic microorganism” refers to light that can be utilized by the microorganism to grow and/or produce carbon-based products of interest, for example, fuels, including biofuels.

As used herein, “transparent” refers to an optical property that allows passage of light of a wavelength that is photosynthetically active in the phototrophic microorganism and or other desirable wavelengths of light.

As used herein, “flexible wall” refers to a sheet or sheets of material that have the ability to flex or bend under a relative force or pressure applied to a surface during operation. In some embodiments of the invention, a channel can be formed by bonding two or more flexible walls collinearly and lengthwise, providing bonded seams. In other embodiments of the invention, a channel can be formed by a single flexible wall in the form of a tube.

As used herein, “thin-film” refers to a flexible film, for example, a polymer or polymer composite film. Thickness of the film or sheet can be less than 500 micrometers, preferably from about 100 to about 250 micrometers.

“Phototrophs” or “photoautotrophs” are organisms that carry out photosynthesis. Such organisms include eukaryotic plants, algae, protists and prokaryotic cyanobacteria, green sulfur bacteria, green non-sulfur bacteria, purple sulfur bacteria, and purple non-sulfur bacteria. Phototrophs include natural and engineered organisms that carry out photosynthesis and hyperlight capturing organisms.

The photobioreactors of the present invention are adapted to support a biologically active environment that allows chemical processes involving photosynthesis in organisms such as phototrophic organisms to be carried out, or biochemically active substances to be derived from such organisms. The photobioreactors can support aerobic or anaerobic organisms.

As used herein, “organisms” encompasses autotrophs, phototrophs, heterotrophs, engineered light capturing organisms and at the cellular level, e.g., unicellular and multicellular.

As used herein, “light” generally refers to sunlight but can be solar or from artificial sources including incandescent lights, light-emitting diodes (LEDs), fiber optics, metal halide, neon, halogen and fluorescent lights.

As used herein, “psi” refers to psig (psi gauge).

### EXEMPLIFICATION

### Example 1

### An Exemplary Photobioreactor Capsule

A photobioreactor capsule was fabricated from two sheets of a multi-layer film. The film was composed of five separate layers with the following construction:
LLDPE|tie|PA-6/6,6|tie|LLDPE,

where LLDPE is linear low density polyethylene, tie refers to a less than 5 μm tie layer used to adhere the two adjacent layers together, typically using an anhydride chemistry, and PA-6/6,6 is a blend of nylon-6 and nylon-6,6 commonly referred to as nylon triple six. The LLDPE and PA-6/6,6 layers contained UV stabilizers for weathering protection and likely other additives for processing and handling characteristics. The total thickness of this multi-layer film was 9 mil (229 μm). Neglecting the thicknesses of the tie layers, the thicknesses of the polyethylene and nylon layers were:
3.6 mil LLDPE|1.8 mil PA-6/6,6|3.6 mil LLDPE.

This multilayer film was made via an extrusion process on a blown film line, giving a collapsed tube. The collapsed tube was slit to the desired film width. Two sheets of the film were joined together either by a thermal, heat sealing process or an extruded sealing process where a PE resin was used to create the channels along the length of the capsule. The transition geometries were fabricated in a secondary step using a thermal, heat sealing process.

Table 3 is a list of the properties of a capsule constructed as described above.

TABLE 3
Properties of capsule film and seams

| Property | | |
|---|---|---|
| Type | Test | Value |
| Mechanical | Film tensile at 22 C., 0% RH, | >20 lb/in at <20% strain |
| | 0.6 min⁻¹ ^{a} | |
| Mechanical | Seam T-Peel tensile at 22 C., | >22 lb/in with >100% strain |
| | 0% RH, 0.6 min⁻¹ ^{b} | |
| Mechanical | Film and seam tensile creep at | <20% strain |
| | constant load of 7 lb/in, 37 C., | |
| | 100% RH | |

^{a} measured using a modified version of ASTM standard D882, the teachings of which are incorporated herein by reference in their entirety.
^{b} measured using a modified version of ASTM standard D1876, the teachings of which are incorporated by reference in their entirety.

### Example 2

### Photobioreactor Capsule Operation at Scale

Photobioreactor capsules 0.4 m wide and either 8 m or 50 m long were constructed of the film described in Example 1 and contained 8 channels with a center to center lay flat width (LFW) of 50 mm and a seal width of 4 mm. The transition region of these capsules had a 10 degree taper leading to a connector attached to the fluid and gas piping. There were no internal seals in the transition area and to manage the high hoop stresses in the film created by the internal pressure, an external pressure plate or “clamshell” was used to constrain the transition area and transfer the loads into the higher stiffness plates. The clamshell also utilized narrow fins that provided a compressive force on the channel seal ends. FIGS. 8 and 9 depict an exemplary pressure plate from several perspectives.

Cell viability and growth have been demonstrated with the photobioreactor capsules using a wild type cyanobacteria as well as a genetically-modified cyanobacteria. The capsules were operated for the times and pressures listed in Table 4.

TABLE 4
Run conditions for 8 channel photobioreactor capsules.

| | Capsule Dimensions | | |
|---|---|---|---|
| Capsule Number | (width × length) | Time Operated | Inlet Pressure |
| 1 | 0.4 m × 50 m | 3 days | 2.30 psi |
| 2 | 0.4 m × 8 m | 27 days | 2.85 psi |
| 3 | 0.4 m × 50 m | 13 days | 1.31 psi |
| 4 | 0.4 m × 50 m | 13 days | 1.38 psi |
| 5 | 0.4 m × 50 m | 7 days | 2.23 psi |
| 6 | 0.4 m × 50 m | 7 days | 2.26 psi |
| 7 | 0.4 m × 50 m | 6 days | 2.08 psi |
| 8 | 0.4 m × 50 m | 6 days | 2.05 psi |
| 9 | 0.4 m × 50 m | 6 days | 4.01 psi |
| 10 | 0.4 m × 50 m | 6 days | 3.91 psi |

### Example 3

### Alternative Transition Area Geometries

Alternative geometries that can be used with or without a pressure plate were evaluated through pressure testing in the lab. The prototypes whose fluid distribution structures are depicted in FIGS. 4A-4G had 8 channels and a total lay flat width of 0.40 m to 0.45 m. FIG. 5D is an illustration of a prototype having 22 channels and a total lay flat width of about 1 m.

FIG. 10 depicts a photobioreactor capsule with an integrated header. Pressure testing of an 8-channel capsule with an integrated header similar to that depicted in FIG. 10 (8 channel capsule with 44 mm inflated diameter header) showed no failure of the capsule up to 7.93 psi. In addition, modeling was done using ANSYS software on a 22 channel capsule where each channel has a 29 mm inflated diameter. The mean flow rate in each channel was 0.3 m/s. FIG. 11 shows that the capsule with the integrated header having a 100 mm inflated diameter had more even channel flow velocity than the capsule with the integrated header having a 50 mm or 75 mm inflated diameter.

Qualitative flow testing data was obtained for capsules including several of the fluid distribution structures depicted herein. The pressure of the liquid flow was measured at the inlet and the outlet of each capsule and is indicated in psi in Tables 5 and 6. In addition, the liquid flow in each channel was ranked with a 0, 1 or 2, where 0 indicated no fluid flow, 1 indicated inhibited fluid flow and 2 indicated full fluid flow. Tables 5-7 show the results of the qualitative flow testing.

Table 5 shows the results of the qualitative flow tests on 17-channel capsules having integrated headers of varying inflated diameters and with or without a c-tube, such as that depicted in FIG. 12. In Table 5, “HughTube” is a c-tube, such as that depicted in FIG. 12, in which the opening of the “c” is oriented at approximately parallel to the ground; “HughTube Rotated” indicates the HughTube has been rotated up and away from the horizontal by about 70 to about 80 degrees.

TABLE 5
Results of Qualitative Flow Distribution Tests on 17-
channel Side-fill Capsules with an Integrated Header.

| | | | 4″ Header | 4″ Header | | | |
|---|---|---|---|---|---|---|---|
| | 4″ | 4″ Header | Side Fill, | Side Fill, | 2″ | 3″ | 3″ |
| Channel | Header | Side Fill, | HughTube | HughTube | Header | Header | Header |
| No. | Side Fill | HughTube | Rotated | Rotated | Side Fill | Side Fill | Side Fill |
| 1 | 1 | 2 | 2 | 2 | 1 | 1 | 1 |
| 2 | 2 | 2 | 2 | 2 | 2 | 1 | 1 |
| 3 | 2 | 2 | 2 | 2 | 2 | 1 | 1 |
| 4 | 2 | 2 | 2 | 2 | 2 | 1 | 1 |
| 5 | 2 | 1 | 1 | 1 | 2 | 1 | 1 |
| 6 | 2 | 1 | 1 | 1 | 2 | 2 | 2 |
| 7 | 2 | 2 | 1 | 1 | 2 | 2 | 2 |
| 8 | 2 | 2 | 1 | 1 | 2 | 2 | 2 |
| 9 | 2 | 2 | 2 | 2 | 1 | 2 | 2 |
| 10 | 2 | 2 | 2 | 2 | 1 | 2 | 2 |
| 11 | 1 | 2 | 2 | 2 | 1 | 2 | 2 |
| 12 | 1 | 2 | 2 | 2 | 2 | 2 | 2 |
| 13 | 1 | 2 | 2 | 2 | 2 | 2 | 2 |
| 14 | 1 | 2 | 2 | 2 | 2 | 2 | 2 |
| 15 | 1 | 2 | 2 | 2 | 2 | 2 | 2 |
| 16 | 1 | 2 | 2 | 2 | 2 | 2 | 2 |
| 17 | 1 | 1 | 2 | 2 | 1 | 2 | 2 |
| Inlet | 0.5 | 0.5 | 0.5 | 1 | 4 | 1.75 | 2 |
| Pressure, | | | | | | | |
| atm | | | | | | | |
| Outlet | 0 | 0 | 0 | 0.5 | 0 | 0 | 0.5 |
| Pressure, | | | | | | | |
| atm | | | | | | | |

Table 6 shows the results of the qualitative flow tests on 8-channel capsules having in-line, tapered fluid distribution structures. In Table 6, “Diamond Transition” refers to the fluid distribution structure depicted in FIG. 4F; “Split Transition” refers to the fluid distribution structure depicted in FIG. 4D; “Split Manifold Transition” refers to the fluid distribution structure depicted in FIG. 4G.

TABLE 6
Results of Qualitative Flow Distribution Tests on 8-channel
Capsules with In-line, Tapered Fluid distribution structures.

| | | | | Split | | |
|---|---|---|---|---|---|---|
| | Diamond | Diamond | Diamond | Manifold | Diamond | Diamond |
| | Transition, | Transition, | Transition, | Transition, | Transition, | Transition, |
| Channel | 4″ | 4″ | 4″ | 2″ | 2″ | 2″ |
| No. | Connector | Connector | Connector | Connector | Connector | Connector |
| 1 | 2 | 2 | 2 | 2 | 2 | 2 |
| 2 | 2 | 2 | 2 | 2 | 2 | 2 |
| 3 | 2 | 2 | 2 | 2 | 2 | 2 |
| 4 | 2 | 2 | 2 | 2 | 2 | 2 |
| 5 | 2 | 2 | 2 | 2 | 2 | 2 |
| 6 | 2 | 2 | 2 | 2 | 2 | 2 |
| 7 | 2 | 2 | 2 | 2 | 2 | 2 |
| 8 | 2 | 2 | 2 | 2 | 2 | 2 |
| Inlet | 1 | 2 | 4 | 5 | 5.8 | 5 |
| Pressure, | | | | | | |
| atm | | | | | | |
| Outlet | 0 | 1.1 | 3 | 0 | 0 | 0 |
| Pressure, | | | | | | |
| atm | | | | | | |
| | Diamond | Spilt | Split | Split | Split | Split |
| | Transition, | Transition, | Transition, | Transition, | Transition, | Transition, |
| Channel | 2″ | 2″ | 4″ | 4″ | 4″ | 4″ |
| No. | Connector | Connector | Connector | Connector | Connector | Connector |
| 1 | 2 | 2 | 2 | 2 | 2 | 2 |
| | | | | Split | | |
| | Diamond | Diamond | Diamond | Manifold | Diamond | Diamond |
| | Transition, | Transition, | Transition, | Transition, | Transition, | Transition, |
| Channel | 4″ | 4″ | 4″ | 2″ | 2″ | 2″ |
| No. | Connector | Connector | Connector | Connector | Connector | Connector |
| 2 | 2 | 2 | 1 | 2 | 2 | 1 |
| 3 | 2 | 2 | 2 | 2 | 2 | 2 |
| 4 | 2 | 2 | 2 | 2 | 2 | 2 |
| 5 | 2 | 2 | 2 | 2 | 2 | 2 |
| 6 | 2 | 2 | 2 | 2 | 2 | 2 |
| 7 | 2 | 2 | 2 | 2 | 2 | 2 |
| 8 | 2 | 2 | 2 | 2 | 1 | 2 |
| Inlet | 6 | 6.1 | 0.8 | 2 | 4 | 0.9 |
| Pressure, | | | | | | |
| atm | | | | | | |
| Outlet | 2.2 | 0 | 0 | 1 | 3 | 0 |
| Pressure, | | | | | | |
| atm | | | | | | |

| | Split | Split | Split | Split | Split |
|---|---|---|---|---|---|
| | Transition, | Transition, | Transition, | Transition, | Transition, |
| Channel | 4″ | 4″ | 4″ | 4″ | 4″ |
| No. | Connector | Connector | Connector | Connector | Connector |
| 1 | 2 | 2 | 1 | 2 | 2 |
| 2 | 1 | 2 | 2 | 2 | 2 |
| 3 | 2 | 2 | 2 | 2 | 2 |
| 4 | 2 | 2 | 2 | 2 | 2 |
| 5 | 2 | 2 | 2 | 2 | 2 |
| 6 | 2 | 2 | 2 | 2 | 2 |
| 7 | 2 | 2 | 2 | 2 | 2 |
| 8 | 2 | 2 | 2 | 2 | 2 |
| Inlet | 2 | 4 | 0.25 | 2 | 4 |
| Pressure, | | | | | |
| atm | | | | | |
| Outlet | 1.1 | 3.1 | 0 | 1 | 3 |
| Pressure, | | | | | |
| atm | | | | | |

TABLE 7
Results of Qualitative Flow Test on 22-channel Capsules with
In-line, Fluid distribution structure Depicted in FIG. 5C.

| Channel No. | 4″ perforated inlet |
|---|---|
| 1 | 1 |
| 2 | 1 |
| 3 | 2 |
| 4 | 2 |
| 5 | 2 |
| 6 | 2 |
| 7 | 2 |
| 8 | 2 |
| 9 | 2 |
| 10 | 2 |
| 11 | 2 |
| 12 | 2 |
| 13 | 2 |
| 14 | 2 |
| 15 | 2 |
| 16 | 2 |
| 17 | 2 |
| 18 | 2 |
| 19 | 2 |
| 20 | 2 |
| 21 | 1 |

The photobioreactor with an integrated header can optionally have a pressure plate designed to manage the pressure in the photobioreactor. FIG. 12 is an example of a C-tube with adjustable plug that is designed to manage pressure and length of the header in a capsule with an integrated header.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of this invention are presented for purposes of illustration and description. They are not intended to be exhaustive or to limit the invention to the precise forms disclosed; obviously many modifications and variations are possible in view of the above teachings. The embodiments were chosen and described in order to best explain the principles of the invention and its practical applications. These procedures will enable others, skilled in the art, to best utilize the invention and various embodiments with various modifications. It is intended that the scope of the invention be defined by the following claims and their equivalents. Modifications and substitutions by one of ordinary skill in the art are considered to be within the scope of the present invention, which is not to be limited except by the following claims.

The relevant teachings of all patents, published applications, and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A photobioreactor for a phototrophic microorganism, and culture medium therefor, comprising a capsule, the capsule comprising:
an elongated body having a length and a width and comprised of a first, flexible polymer film that is at least partially transparent to light of a wavelength that is photosynthetically active in a phototrophic microorganism, wherein the elongated body is divided widthwise into a plurality of adjacent channels, each having a major cross-sectional dimension that is not more than about one-third of the length of the elongated body, when the elongated body is inflated; and
a tapered fluid distribution structure coupled to the elongated body adapted for fluid communication with the plurality of adjacent channels that distributes a flow of culture medium amongst the plurality of channels, wherein the tapered fluid distribution structure is comprised of a second, flexible polymer film.

2. (canceled)

3. The photobioreactor of claim 1, wherein the elongated body includes a top wall composed of the flexible polymer film at least partially transparent to light of a wavelength that is photosynthetically active in a phototrophic microorganism and a bottom wall, wherein the top wall and the bottom wall are sealed lengthwise to form one or more seams that form the plurality of channels in the elongated body.

4. The photobioreactor of claim 3, wherein the tapered fluid distribution structure includes a top wall and a bottom wall that are sealed to one another to form the tapered fluid distribution structure.

5. The photobioreactor of claim 4, wherein the top wall of the elongated body and the top wall of the tapered fluid distribution structure, together, are formed from a single piece of flexible polymer film, and the bottom wall of the elongated body and the bottom wall of the tapered fluid distribution structure, together, are formed form a second, single piece of flexible polymer film.

6.-7. (canceled)

8. The photobioreactor of claim 5, wherein the top and bottom walls of the elongated body are sealed lengthwise at even intervals to form two or more evenly-spaced seams.

9.-11. (canceled)

12. The photobioreactor of claim 8, wherein the length of the elongated body is at least about 50 m and the major cross-sectional dimension of each channel is between about 10 mm and 100 mm, inclusive, when the elongated body is inflated.

13. The photobioreactor of claim 12, wherein the tapered fluid distribution structure is in the form of an integrated header.

14. (canceled)

15. The photobioreactor of claim 12, wherein the first, flexible polymer film and the second, flexible polymer film are the same.

16. The photobioreactor of claim 12, wherein the tapered fluid distribution structure includes a series of barriers adapted and positioned to support distribution of the flow of culture medium amongst the plurality of channels.

17. The photobioreactor of claim 16, wherein the tapered fluid distribution structure includes a top wall and a bottom wall and the barriers are provided by sealing a portion of the top wall to a portion of the bottom wall to form seams.

18. The photobioreactor of claim 17, wherein the one or more seams of the tapered fluid distribution structure and the second, flexible polymer film are designed for operation at a pressure of at least 2 psi with a factor of safety of at least three.

19. The photobioreactor of claim 18, wherein the one or more seams of the tapered fluid distribution structure and the second, flexible polymer film are designed for operation at a pressure of at least 4 psi with a factor of safety of at least three.

20.-32. (canceled)

33. A photobioreactor for a phototrophic microorganism, and culture medium therefor, comprising a capsule, the capsule comprising:
an elongated body divided widthwise into a plurality of adjacent channels, the elongated body having a length of at least 50 m and a width of about 1 m to about 2 m, inclusive, and including a first top wall and a first bottom wall sealed lengthwise to form two or more seams that form the plurality of adjacent channels; and
a tapered fluid distribution structure including a second top wall coupled to the first top wall and a second bottom wall coupled to the first bottom wall,
wherein the first and second top walls are formed from a first single piece of a flexible polymer composite film and the first and second bottom walls are formed from a second single piece of the flexible polymer composite film, the flexible polymer composite film is at least partially transparent to light of a wavelength that is photosynthetically active in a phototrophic microorganism, and each channel has a major cross-sectional dimension of between about 25 mm and about 50 mm, inclusive.

34. An array, comprising a plurality of the photobioreactors of claim 1.

35.-38. (canceled)

39. A method of producing a carbon-based product, comprising culturing a phototrophic microorganism that photosynthetically produces the carbon-based product in the photobioreactor of claim 1, the method comprising:
flowing culture medium containing the phototrophic microorganism into and through the tapered fluid distribution structure at an operating pressure of greater than about 2 psi, thereby distributing culture medium to the plurality of adjacent channels;
flowing the distributed culture medium through the plurality of adjacent channels;
providing carbon dioxide to the culture medium;
providing light of a wavelength that is photosynthetically active in the phototrophic microorganism to the phototrophic microorganism as it flows through the plurality of adjacent channels, whereby the phototrophic microorganism produces the carbon-based product; and
collecting the carbon-based product.

40. A method of culturing a phototrophic microorganism in the photobioreactor of claim 1, the method comprising:
flowing culture medium containing the phototrophic microorganism into and through the tapered fluid distribution structure at an operating pressure of greater than about 2 psi, thereby distributing culture medium across the plurality of adjacent channels;
flowing the distributed culture medium through the plurality of adjacent channels;
providing carbon dioxide to the culture medium; and
providing light of a wavelength that is photosynthetically active in the phototrophic microorganism to the phototrophic microorganism as it flows through the plurality of adjacent channels,
thereby culturing the phototrophic microorganism.

41. The method claim 40, wherein the phototrophic microorganisms and culture medium are distributed to a substantially even depth across the plurality of adjacent channels by seams in the tapered fluid distribution structure positioned to provide alternating sequences of separated channel flows and un-separated channel flows.

42. The method of claim 41, wherein each un-separated channel flow is re-distributed into two or more channel flows by additional seams in the tapered fluid distribution structure.

43. The method of claim 40, wherein each channel, when filled with culture medium, has a substantially semi-circular cross-section.

44. (canceled)

45. The method of culturing phototrophic microorganisms of claim 40, wherein the substantially even depth across the plurality of adjacent channels averages between about 5 mm to about 30 mm.

46.-58. (canceled)
